SEQUENCE LISTING (54 sequences)

SEQ ID NO: 1 - protein, 147 aa, UNKNOWN
  Marine metagenome
MKISLEGLSLIKKFEGCKLEAYKCSAGVWTIGYGHTAGVKEGDVCTQEEAEKLLRGDIFKFEEYVQDSVKVDLDQSQFDALVAWTFNLGPGNLRSSTMLKKLNNGEYESVPFEMRRWNKAGGKTLDGLIRRRQAESLLFESKEWHQV

SEQ ID NO: 2 - protein, 143 aa, Pseudomonas putida
MRTSQRGLSLIKSFEGLRLQAYQDSVGVWTIGYGTTRGVKAGMKISKDQAERMLLNDVQRFEPEVERLIKVPLNQDQWDALMSFTYNLGAANLESSTLRRLLNAGNYAAAAEQFPRWNKAGGQVLAGLTRRRAAERELFLGAA

SEQ ID NO: 3 - protein, 144 aa, Pseudomonas phage PAJU2
MRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLKLLNKGDYQGAADQFPRWVNAGGKRLDGLVKRRAAERALFLEPLS

SEQ ID NO: 4 - protein, 144 aa, Artificial Sequence
  SYNTHETIC POLYPEPTIDE
MRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 5 - protein, 143 aa, Artificial Sequence
  SYNTHETIC POLYPEPTIDE
MRTSQRGLSLIKSFEGLRLQAYQDSVGVWTIGYGTTRGVKAGMKISKDQAERMLLNDVQRFEPEVERLIKVPLNQDQWDALMSFTYNLGAANLESSTLRDLLNAGNYAAAAEQFPHWNKAGGQVLAGLTRRRAAERELFLGAA

SEQ ID NO: 6 - protein, 158 aa, ARTIFICIAL SEQUENCE
  SYNTHETIC POLYPEPTIDE
GPRRPRRPGRRAPVMRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLKLLNKGDYQGAADQFPRWVNAGGKRLDGLVKRRAAERALFLEPLS

SEQ ID NO: 7 - protein, 172 aa, ARTIFICIAL SEQUENCE
  SYNTHETIC POLYPEPTIDE
KFFKFFKFFKAGAGAGAGAGAGAGAGASMRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLKLLNKGDYQGAADQFPRWVNAGGKRLDGLVKRRAAERALFLEPLS

SEQ ID NO: 8 - protein, 171 aa, ARTIFICIAL SEQUENCE
  SYNTHETIC POLYPEPTIDE
KRKKRKKRKAGAGAGAGAGAGAGAGASMRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLKLLNKGDYQGAADQFPRWVNAGGKRLDGLVKRRAAERALFLEPLS

SEQ ID NO: 9 - protein, 158 aa, ARTIFICIAL SEQUENCE
  SYNTHETIC POLYPEPTIDE
MRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLSGPRRPRRPGRRAPV

SEQ ID NO: 10 - protein, 181 aa, Pseudomonas phage Lu11
MNNELPWVAEARKYIGLREDTSKTSHNPKLLAMLDRMGEFSNESRAWWHDDETPWCGLFVGYCLGVAGRYVVREWYRARAWEAPQLTKLDRPAYGALVTFTRSGGGHVGFIVGKDARGNLMVLGGNQSNAVSIAPFAVSRVTGYFWPSFWRNKTAVKSVPFEERYSLPLLKSNGELSTNEA

SEQ ID NO: 11 - protein, 439 aa, Pseudomonas sp.
MKRTTLNLELESNTDRLLQEKDDLLPQSVTNSSDEGTPFAQVEGASDDNTAEQDSDKPGASVADADTKPVDPEWKTITVASGDTLSTVFTKAGLSTSAMHDMLTSSKDAKRFTHLKVGQEVKLKLDPKGELQALRVKQSELETIGLDKTDKGYSFKREKAQIDLHTAYAHGRITSSLFVAGRNAGLPYNLVTSLSNIFGYDIDFALDLREGDEFDVIYEQHKVNGKQVATGNILAARFVNRGKTYTAVRYTNKQGNTSYYRADGSSMRKAFIRTPVDFARISSRFSLGRRHPILNKIRAHKGVDYAAPIGTPIKATGDGKILEAGRKGGYGNAVVIQHGQRYRTIYGHMSRFAKGIRAGTSVKQGQIIGYVGMTGLATGPHLHYEFQINGRHVDPLSAKLPMADPLGGADRKRFMAQTQPMIARMDQEKKTLLALNKQR

SEQ ID NO: 12 - protein, 126 aa, Micavibrio aeruginosavorus
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYR

SEQ ID NO: 13 - protein, 144 aa, ARTIFICIAL SEQUENCE
  SYNTHETIC POLYPEPTIDE
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYRRKKTRKRLKKIGKVLKWI

SEQ ID NO: 14 - protein, 154 aa, ARTIFICIAL SEQUENCE
  SYNTHETIC POLYPEPTIDE
KFFKFFKFFKAGAGAGAGAGAGAGAGASMTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYR

SEQ ID NO: 15 - protein, 157 aa, ARTIFICIAL SEQUENCE
  SYNTHETIC POLYPEPTIDE
MRTSQRGLSLIKSFEGLRLQAYQDSVGVWTIGYGTTRGVKAGMKISKDQAERMLLNDVQRFEPEVERLIKVPLNQDQWDALMSFTYNLGAANLESSTLRDLLNAGNYAAAAEQFPHWNKAGGQVLAGLTRRRAAERELFLGAAGPRRPRRPGRRAPV

SEQ ID NO: 16 - protein, 157 aa, ARTIFICIAL SEQUENCE
  SYNTHETIC POLYPEPTIDE
GPRRPRRPGRRAPVMRTSQRGLSLIKSFEGLRLQAYQDSVGVWTIGYGTTRGVKAGMKISKDQAERMLLNDVQRFEPEVERLIKVPLNQDQWDALMSFTYNLGAANLESSTLRRLLNAGNYAAAAEQFPRWNKAGGQVLAGLTRRRAAERELFLGAA

SEQ ID NO: 17 - protein, 150 aa, ARTIFICIAL SEQUENCE
  SYNTHETIC POLYPEPTIDE
MSFKLGKRSLSNLEGVHPDLIKVVKRAIELTECDFTVTEGLRSKERQAQLLKEKKTTTSNSRHLTGHAVDLAAWVNNTVSWDWKYYYQIADAMKKAASELNVSIDWGGDWKKFKDGPHFELTWSKYPIKGASRKKTRKRLKKIGKVLKWI

SEQ ID NO: 18 - protein, 134 aa, ARTIFICIAL SEQUENCE
  SYNTHETIC POLYPEPTIDE
MKLSEKRALFTQLLAQLILWAGTQDRVSVALDQVKRTQAEADANAKSGAGIRNSLHLLGLAGDLILYKDGKYMDKSEDYKFLGDYWKSLHPLCRWGGDFKSRPDGNHFSLEHEGVQRKKTRKRLKKIGKVLKWI

SEQ ID NO: 19 - protein, 132 aa, Acinetobacter sp.
MSFKLGKRSLSNLEGVHPDLIKVVKRAIELTECDFTVTEGLRSKERQAQLLKEKKTTTSNSRHLTGHAVDLAAWVNNTVSWDWKYYYQIADAMKKAASELNVSIDWGGDWKKFKDGPHFELTWSKYPIKGAS

SEQ ID NO: 20 - protein, 116 aa, Pseudomonas phage PaP2
MKLSEKRALFTQLLAQLILWAGTQDRVSVALDQVKRTQAEADANAKSGAGIRNSLHLLGLAGDLILYKDGKYMDKSEDYKFLGDYWKSLHPLCRWGGDFKSRPDGNHFSLEHEGVQ

SEQ ID NO: 21 - protein, 157 aa, UNKNOWN
  Marine metagenome
MKNFNEIIEHVLKHEGGYVNDPKDLGGETKYGITKRFYPDLDIKNLTIEQATEIYKKDYWDKNKVESLPQNLWHIYFDMCVNMGKRTAVKVLQRAAVNRGRDIEVDGGLGPATIGALKGVELDRVRAFRVKYYVDLITARPEQEKFYLGWFRRATEV

SEQ ID NO: 22 - protein, 231 aa, Pseudomonas phage phi2954
MSKQGGVKVAQAVAALSSPGLKIDGIVGKATRAAVSSMPSSQKAATDKILQSAGIGSLDSLLAEPAAATSDTFREVVLAVAREARKRGLNPAFYVAHIALETGWGRSVPKLPDGRSSYNYAGLKYAAVKTQVKGKTETNTLEYIKSLPKTVRDSFAVFASAGDFSRVYFWYLLDSPSAYRYPGLKNAKTAQEFGDILQKGGYATDPAYAAKVASIASTAVARYGSDVSSVA

SEQ ID NO: 23 - protein, 202 aa, Pseudomonas phage Lu11
MSDKRVEITGNVSGFFESGGRGVKTVSTGKGDNGGVSYGKHQLASNNGSMALFLESPFGAPYRAQFAGLKPGTAAFTSVYNKIANETPTAFERDQFQYIAASHYDPQAAKLKAEGINVDDRHVAVRECVFSVAVQYGRNTSIIIKALGSNFRGSDKDFIEKVQDYRGATVNTYFKSSSQQTRDSVKNRSQQEKQMLLKLLNS

SEQ ID NO: 24 - protein, 268 aa, Pseudomonas aeruginosa
MTLRYGDRSQEVRQLQRRLNTWAGANLYEDGHFGAATEDAVRAFQRSHGLVADGIAGPKTLAALGGADCSHLLQNADLVAAATRLGLPLATIYAVNQVESNGQGFLGNGKPAILFERHIMYRRLAAHDQVTADQLAAQFPALVNPRPGGYAGGTAEHQRLANARQIDDTAALESASWGAFQIMGFHWQRLGYISVQAFAEAMGRSESAQFEAFVRFIDTDPALHKALKARKWADFARLYNGPDYKRNLYDNKLARAYEQHANCAEASA

SEQ ID NO: 25 - protein, 160 aa, Pseudomonas aeruginosa
MAVVSEKTAGGRNVLAFLDMLAWSEGTSTIRGSDNGYNVVVGGGLFNGYADHPRLKVYLPRYKVYSTAAGRYQLLSRYWDAYRESLALKGGFTPSNQDLVALQQIKERRSLADIQAGRLADAVQKCSNIWASLPGAGYGQREHSLDDLTAHYLAAGGVLS

SEQ ID NO: 26 - protein, 185 aa, Acinetobacter phage phiAB6
MILTKDGFSIIRNELFEGKLDQTQVDAINFIVEKATEYGLTYPEAAYLLATIYHETGLPSGYRTMQPIKEAGSDSYLRSKKYYPYIGYGYVQLTWEENYERIGKLIGIDLVKNPEKALEPLIAIQIAIKGMLNGWFTGVGFRRKRPVSKYNKQQYVAARNIINGKDKAELIAKYAIIFERALRSL

SEQ ID NO: 27 - protein, 266 aa, Pseudomonas phage PhiPA3
MTLLKKGDKGDAVKQLQQKLKDLGYTLGVDGNFGNGTDTVVRSFQTKMKLSVDGVVGNGTMSTIDSTLAGIKAWKTSVPFPATNKSRAMAMPTLTEIGRLTNVDPKLLATFCSIESAFDYTAKPYKPDGTVYSSAEGWFQFLDATWDDEVRKHGKQYSFPVDPGRSLRKDPRANGLMGAEFLKGNAAILRPVLGHEPSDTDLYLAHFMGAGGAKQFLMADQNKLAAELFPGPAKANPNIFYKSGNIARTLAEVYAVLDAKVAKHRA

SEQ ID NO: 28 - protein, 14 aa, ARTIFICIAL SEQUENCE
  SYNTHETIC POLYPEPTIDE
GPRRPRRPGRRAPV

SEQ ID NO: 29 - protein, 10 aa, ARTIFICIAL SEQUENCE
  SYNTHETIC POLYPEPTIDE
KFFKFFKFFK

SEQ ID NO: 30 - protein, 9 aa, ARTIFICIAL SEQUENCE
  SYNTHETIC POLYPEPTIDE
KRKKRKKRK

SEQ ID NO: 31 - protein, 18 aa, ARTIFICIAL SEQUENCE
  SYNTHETIC POLYPEPTIDE
AGAGAGAGAGAGAGAGAS

SEQ ID NO: 32 - protein, 18 aa, ARTIFICIAL SEQUENCE
  SYNTHETIC POLYPEPTIDE
RKKTRKRLKKIGKVLKWI

SEQ ID NO: 33 - DNA, 432 bp, pseudomonas putida
atgcggacatcgcaacgcggcttgagcctcatcaagtcgttcgagggcctgcgtctgcaggcatatcaagattcagtaggcgtctggacgattggctacgggaccactcgtggcgtgaaggccggcatgaaaatcagcaaggaccaggcagagcgcatgctgctgaacgacgtgcagcgcttcgagcctgaagttgagcgcctgatcaaggtgccgctgaatcaggatcagtgggacgccctgatgagcttcacctacaacctgggggcggcaaacctcgaatcgtccacgctccgccgactgctcaatgctggcaactacgcagctgctgctgagcagttcccgcgctggaacaaggctggcgggcaagtacttgccggcctaacccgtcggcgtgcagctgagcgggagctgttcctgggggccgcgtga

SEQ ID NO: 34 - DNA, 609 bp, Pseudomonas phage Lu11
atgtctgataaacgcgttgaaattaccggaaacgtttccggttttttcgagtccggtggccgtggtgtaaaaaccgtttctaccggcaaaggtgacaacggcggtgtgagctacggcaagcatcagctggcgtcgaataacggctctatggctctgttccttgaatctccgttcggtgctccgtaccgtgcgcaattcgcaggactgaaaccgggaaccgctgcgtttacttccgtgtacaacaaaatcgcaaatgaaacgccgaccgcgtttgaacgggaccagttccaatacatcgcggcttcgcactacgatccacaagcggccaagctgaaagccgaaggcattaacgtcgatgaccgacatgtcgcggtgcgtgaatgcgtgttcagcgtagccgtgcaatatggtcgaaatacttcgatcattatcaaagcactcggcagtaatttccggggcagcgacaaagacttcatcgaaaaggtgcaggactatcgcggtgccacggttaacacctactttaaatccagtagccagcaaactcgcgacagcgtgaaaaaccgctcgcagcaagaaaagcaaatgctgctgaaactcctgaatagttaa

SEQ ID NO: 35 - DNA, 807 bp, Pseudomonas aeruginosa
atgacccttcgatatggtgatcgttctcaagaggtccgccagcttcagcgtcgactgaacacctgggccggcgccaacctctacgaggacggccacttcggcgccgccactgaggacgcggtgcgcgccttccagcgctcgcatggcctggtcgccgatggcatcgccggcccgaagaccctggccgctctcggcggagctgactgctcgcacctgctgcagaacgccgacctcgtcgccgccgcaactcgcctcggcctgccgctggcgacgatctatgcggtcaatcaggtcgagtcgaacggccaggggttcctgggcaacggcaagccggcaatcctgttcgaacgccacatcatgtaccgccgtctcgccgcccacgatcaggtcaccgccgaccagttggccgcacagttccccgcgctggtgaatcctcgcccgggcggctatgccggcggaaccgccgagcaccagcgcctggcgaacgctcgccagatcgacgataccgccgcactggagtcggccagttggggagccttccagatcatgggtttccactggcaacgcctgggctacatcagcgtgcaggccttcgccgaggccatggggcgcagcgagtcggctcagttcgaagcgttcgtccgcttcatcgacaccgacccggcgctacacaaggcgctgaaggctcgcaaatgggccgacttcgcccgcctctacaatggccccgactacaagcggaacctctacgacaacaagctcgcgcgggcctacgagcaacacgccaactgcgccgaggccagcgcgtga

SEQ ID NO: 36 - DNA, 474 bp, Artificial Sequence
  SYNTHETIC NUCLEIC ACID
atgaaaaattttaatgaaataattgaacatgttttaaaacatgagggtggttatgtaaatgaccctaaagatttaggtggtgaaaccaagtatggtatcactaaaaggttttatccagatcttgatattaagaatctaacaatagaacaagcaacagaaatctataaaaaagattattgggataaaaacaaagtagaatctcttcctcaaaatctatggcacatttattttgatatgtgtgttaatatgggtaagagaactgcagttaaagttctacaaagagcagctgtcaataggggtagagatatagaagttgatggcggtttaggaccagcgacaatcggagctctcaaaggtgtagaattagatagagttagagctttcagagtaaagtattatgtggatttaataacagctagaccagaacaagagaaattttatttaggatggtttagaagagcaactgaagtataa

SEQ ID NO: 37 - DNA, 696 bp, Pseudomonas phage phi2954
atgagcaaacaaggcggcgtgaaagttgcacaggcagtagccgcgctgtcttcgcctggtctcaaaatcgacggtatcgtcggtaaagcgactcgggcggctgtgtcatcgatgcctagcagccagaaggcggctacggataagatactgcaaagcgctggaattggatcgcttgactccctcttggctgagccggcagcagcgacgtccgataccttccgcgaagtggtgcttgccgttgcgcgtgaggcaagaaaacggggtctaaatcccgctttctatgtggctcacatcgcgttggaaactgggtgggggcgttccgtcccgaaactccctgacgggcgttccagttacaactacgcagggctcaaatatgcggcggttaagacgcaggttaagggtaaaaccgaaaccaatactcttgaatatatcaagagcctaccgaagacggtgcgagactctttcgcagtgtttgcttcggcaggggatttttcgagggtgtacttctggtacctcctcgacagtccgtctgcttatcggtaccccggtctcaagaatgcgaagacagctcaggagtttggtgacatcctccaaaagggcggctacgcgactgacccggcatacgccgcaaaagtagcgtcgatcgcaagcaccgccgtggctcgctacggtagtgatgtgagttccgttgcatag

SEQ ID NO: 38 - DNA, 483 bp, Pseudomonas aeruginosa
atggctgttgtttctgaaaaaaccgctggtggtcgtaacgttctggctttcctggacatgctggcttggtctgaaggtacctctaccatccgtggttctgacaacggttacaacgttgttgttggtggtggtctgttcaacggttacgctgaccacccgcgtctgaaagtttacctgccgcgttacaaagtttactctaccgctgctggtcgttaccagctgctgtctcgttactgggacgcttaccgtgaatctctggctctgaaaggtggtttcaccccgtctaaccaggacctggttgctctgcagcagatcaaagaacgtcgttctctggctgacatccaggctggtcgtctggctgacgctgttcagaaatgctctaacatctgggcttctctgccgggtgctggttacggtcagcgtgaacactctctggacgacctgaccgctcactacctggctgctggtggtgttctgtcttaa

SEQ ID NO: 39 - DNA, 558 bp, Artificial Sequence
  SYNTHETIC NUCLEIC ACID
atgatcctgaccaaagacggtttctctatcatccgtaacgaactgttcgaaggtaaactggaccagacccaggttgacgctatcaacttcatcgttgaaaaagctaccgaatacggtctgacctacccggaagctgcttacctgctggctaccatctaccacgaaaccggtctgccgtctggttaccgtaccatgcagccgatcaaagaagctggttctgactcttacctgcgttctaaaaaatactacccgtacatcggttacggttacgttcagctgacctgggaagaaaactacgaacgtatcggtaaactgatcggtatcgacctggttaaaaacccggaaaaagctctggaaccgctgatcgctatccagatcgctatcaaaggtatgctgaacggttggttcaccggtgttggtttccgtcgtaaacgtccggtttctaaatacaacaaacagcagtacgttgctgctcgtaacatcatcaacggtaaagacaaagctgaactgatcgctaaatacgctatcatcttcgaacgtgctctgcgttctctgtaa

SEQ ID NO: 40 - DNA, 798 bp, Pseudomonas phage PhiPA3
atgacattactgaagaaaggcgacaagggtgacgccgtaaaacaactacagcagaaactcaaagaccttgggtataccctgggtgtcgatggcaacttcggtaatggcaccgatactgtcgttcgttctttccaaaccaaaatgaagcttagtgttgatggtgtggttggtaatggtactatgagtactattgactctactctagcaggcattaaagcgtggaagactagtgtacctttccctgcgacgaacaaatcccgagcaatggcaatgccaacgttgactgaaataggtcgactgacaaacgttgatcctaaattgctagcgacattctgttctatcgaaagcgcgtttgattacacagctaaaccctacaagcccgatggcacagtgtacagctccgccgaaggttggttccagttcctggatgcaacatgggatgacgaagtgcgtaaacacggtaagcaatatagcttccctgttgatcctggtcgttctttgcgtaaagatccacgggctaatggcttgatgggcgctgagttcctcaaagggaatgctgctattctgcggccagtactgggtcatgaaccgagcgacacagatctttatctagcccatttcatgggagcaggtggcgcaaaacagttccttatggccgatcaaaataaattggctgccgaattgttccctggtccagctaaggctaatcctaacatcttctataaatccggaaatattgcccgcactttagcagaggtctatgcagtcctcgatgctaaggtagccaagcatagagct

SEQ ID NO: 41 - DNA, 399 bp, Acinetobacter sp. 1294596
atgtctttcaaactgggtaaacgttctctgtctaacctggaaggtgttcacccggacctgatcaaagttgttaaacgtgctatcgaactgaccgaatgcgacttcaccgttaccgaaggtctgcgttctaaagaacgtcaggctcagctgctgaaagaaaaaaaaaccaccacctctaactctcgtcacctgaccggtcacgctgttgacctggctgcttgggttaacaacaccgtttcttgggactggaaatactactaccagatcgctgacgctatgaaaaaagctgcttctgaactgaacgtttctatcgactggggtggtgactggaaaaaattcaaagacggtccgcacttcgaactgacctggtctaaatacccgatcaaaggtgcttcttaa

SEQ ID NO: 42 - DNA, 351 bp, Pseudomonas phage PaP2
atgaaactcagcgaaaaacgagcactgttcacccagctgcttgcccagttaattctttgggcaggaactcaggatcgagtgtcagtagccttggatcaagtgaaaaggacacaggctgaagctgatgccaatgctaagtctggagcaggcattaggaactctctccatctactgggattagccggtgatcttatcctctacaaggatggtaaatacatggataagagcgaggattataagttcctgggagattactggaagagtctccatcctctttgtcggtggggcggagattttaaaagccgtcctgatggtaatcatttctccttggaacacgaaggagtgcaataa

SEQ ID NO: 43 - DNA, 432 bp, ARTIFICIAL SEQUENCE
  SYNTHETIC NUCLEIC ACID
atgcggacatcgcaacgcggcttgagcctcatcaagtcgttcgagggcctgcgtctgcaggcatatcaagattcagtaggcgtctggacgattggctacgggaccactcgtggcgtgaaggccggcatgaaaatcagcaaggaccaggcagagcgcatgctgctgaacgacgtgcagcgcttcgagcctgaagttgagcgcctgatcaaggtgccgctgaatcaggatcagtgggacgccctgatgagcttcacctacaacctgggggcggcaaacctcgaatcgtccacgctccgcgacctgctcaatgctggcaactacgcagctgctgctgagcagttcccgcattggaacaaggctggcgggcaagtacttgccggcctaacccgtcggcgtgcagctgagcgggagctgttcctgggggccgcgtga

SEQ ID NO: 44 - DNA, 435 bp, ARTIFICIAL SEQUENCE
  SYNTHETIC NUCLEIC ACID
atgcgtacatcccaacgaggcatcgacctcatcaaatccttcgagggcctgcgcctgtccgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcgacctgctgaacaagggtgactaccagggagcagcggaccagttcccgcattgggtgaatgcgggcggtaagcgcttggatggtctggttaagcgtcgagcagccgagcgtgcgctgttcctggagccactatcgtga

SEQ ID NO: 45 - DNA, 474 bp, ARTIFICIAL SEQUENCE
  SYNTHETIC NUCLEIC ACID
atgcgtacatcccaacgaggcatcgacctcatcaaatccttcgagggcctgcgcctgtccgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcaagctgctgaacaagggtgactaccagggagcagcggaccagttcccgcgctgggtgaatgcgggcggtaagcgcttggatggtctggttaagcgtcgagcagccgagcgtgcgctgttcctggagccactatcgggcccccgccggccacgacgacctggacgccgggcacctgtc

SEQ ID NO: 46 - DNA, 519 bp, ARTIFICIAL SEQUENCE
  SYNTHETIC NUCLEIC ACID
aaattctttaagttctttaagttttttaaagccggcgcaggagctggtgcaggagctggtgcaggagctggtgcaggagctagcatgcgtacatcccaacgaggcatcgacctcatcaaatccttcgagggcctgcgcctgtccgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcaagctgctgaacaagggtgactaccagggagcagcggaccagttcccgcgctgggtgaatgcgggcggtaagcgcttggatggtctggttaagcgtcgagcagccgagcgtgcgctgttcctggagccactatcgtaa

SEQ ID NO: 47 - DNA, 516 bp, ARTIFICIAL SEQUENCE
  SYNTHETIC NUCLEIC ACID
aaacgcaagaaacgtaagaaacgcaaagccggcgcaggagctggtgcaggagctggtgcaggagctggtgcaggagctagcatgcgtacatcccaacgaggcatcgacctcatcaaatccttcgagggcctgcgcctgtccgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcaagctgctgaacaagggtgactaccagggagcagcggaccagttcccgcgctgggtgaatgcgggcggtaagcgcttggatggtctggttaagcgtcgagcagccgagcgtgcgctgttcctggagccactatcgtaa

SEQ ID NO: 48 - DNA, 432 bp, ARTIFICIAL SEQUENCE
  SYNTHETIC NUCLEIC ACID
atgacctacaccctgtctaaacgttctctggacaacctgaaaggtgttcacccggacctggttgctgttgttcaccgtgctatccagctgaccccggttgacttcgctgttatcgaaggtctgcgttctgtttctcgtcagaaagaactggttgctgctggtgcttctaaaaccatgaactctcgtcacctgaccggtcacgctgttgacctggctgcttacgttaacggtatccgttgggactggccgctgtacgacgctatcgctgttgctgttaaagctgctgctaaagaactgggtgttgctatcgtttggggtggtgactggaccaccttcaaagacggtccgcacttcgaactggaccgttctaaataccgtaaaaaaacccgtaaacgtctgaaaaaaatcggtaaagttctgaaatggatctaa

SEQ ID NO: 49 - DNA, 465 bp, ARTIFICIAL SEQUENCE
  SYNTHETIC NUCLEIC ACID
aaattctttaagttctttaagttttttaaagccggcgcaggagctggtgcaggagctggtgcaggagctggtgcaggagctagcatgacatacaccctgagcaaaagaagcctggataacctaaaaggcgttcatcccgatctggttgccgttgtccatcgcgccatccagcttacaccggttgatttcgcggtgatcgaaggcctgcgctccgtatcccgccaaaaggaactggtggccgccggcgccagcaagaccatgaacagccgacacctgacaggccatgcggttgatctagccgcttacgtcaatggcatccgctgggactggcccctgtatgacgccatcgccgtggctgtgaaagccgcagcaaaggaattgggtgtggccatcgtgtggggcggtgactggaccacgtttaaggatggcccgcactttgaactggatcggagcaaatacagataa

SEQ ID NO: 50 - DNA, 453 bp, ARTIFICIAL SEQUENCE
  SYNTHETIC NUCLEIC ACID
atgtctttcaaactgggtaaacgttctctgtctaacctggaaggtgttcacccggacctgatcaaagttgttaaacgtgctatcgaactgaccgaatgcgacttcaccgttaccgaaggtctgcgttctaaagaacgtcaggctcagctgctgaaagaaaaaaaaaccaccacctctaactctcgtcacctgaccggtcacgctgttgacctggctgcttgggttaacaacaccgtttcttgggactggaaatactactaccagatcgctgacgctatgaaaaaagctgcttctgaactgaacgtttctatcgactggggtggtgactggaaaaaattcaaagacggtccgcacttcgaactgacctggtctaaatacccgatcaaaggtgcttctcgtaaaaaaacccgtaaacgtctgaaaaaaatcggtaaagttctgaaatggatctaa

SEQ ID NO: 51 - DNA, 474 bp, ARTIFICIAL SEQUENCE
  SYNTHETIC NUCLEIC ACID
ggtccgcgtcgtccgcgtcgtccgggtcgtcgtgctccggttatgcggacatcgcaacgcggcttgagcctcatcaagtcgttcgagggcctgcgtctgcaggcatatcaagattcagtaggcgtctggacgattggctacgggaccactcgtggcgtgaaggccggcatgaaaatcagcaaggaccaggcagagcgcatgctgctgaacgacgtgcagcgcttcgagcctgaagttgagcgcctgatcaaggtgccgctgaatcaggatcagtgggacgccctgatgagcttcacctacaacctgggggcggcaaacctcgaatcgtccacgctccgcgacctgctcaatgctggcaactacgcagctgctgctgagcagttcccgcattggaacaaggctggcgggcaagtacttgccggcctaacccgtcggcgtgcagctgagcgggagctgttcctgggggccgcgtga

SEQ ID NO: 52 - DNA, 480 bp, ARTIFICIAL SEQUENCE
  SYNTHETIC NUCLEIC ACID
ggtccgcgtcgtccgcgtcgtccgggtcgtcgtgctccggttatgcgtacatcccaacgaggcatcgacctcatcaaatccttcgagggcctgcgcctgtcatgcgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcgacctgctgaacaagggtgactaccagggagcagcggaccagttcccgcattgggtgaatgcgggcggtaagcgcttggatggtctggttaagcgtcgagcagccgagcgtgcgctgttcctggagccactatcgtga

SEQ ID NO: 53 - DNA, 405 bp, ARTIFICIAL SEQUENCE
  SYNTHETIC NUCLEIC ACID
atgaaactcagcgaaaaacgagcactgttcacccagctgcttgcccagttaattctttgggcaggaactcaggatcgagtgtcagtagccttggatcaagtgaaaaggacacaggctgaagctgatgccaatgctaagtctggagcaggcattaggaactctctccatctactgggattagccggtgatcttatcctctacaaggatggtaaatacatggataagagcgaggattataagttcctgggagattactggaagagtctccatcctctttgtcggtggggcggagattttaaaagccgtcctgatggtaatcatttctccttggaacacgaaggagtgcaacgtaaaaaaacccgtaaacgtctgaaaaaaatcggtaaagttctgaaatggatctaa

SEQ ID NO: 54 - DNA, 432 bp, ARTIFICIAL SEQUENCE
  SYNTHETIC NUCLEIC ACID
atgcggacatcgcaacgcggcttgagcctcatcaagtcgttcgagggcctgcgtctgcaggcatatcaagattcagtaggcgtctggacgattggctacgggaccactcgtggcgtgaaggccggcatgaaaatcagcaaggaccaggcagagcgcatgctgctgaacgacgtgcagcgcttcgagcctgaagttgagcgcctgatcaaggtgccgctgaatcaggatcagtgggacgccctgatgagcttcacctacaacctgggggcggcaaacctcgaatcgtccacgctccgcgacctgctcaatgctggcaactacgcagctgctgctgagcagttcccgcactggaacaaggctggcgggcaagtacttgccggcctaacccgtcggcgtgcagctgagcgggagctgttcctgggggccgcgtga

(19) United States
(12) Patent Application Publication
(10) Pub. No.: US 2022/0160843 A1
(43) Pub. Date: May 26, 2022

(54) LYSINS AND DERIVATIVES THEREOF WITH BACTERICIDAL ACTIVITY AGAINST PSEUDOMONAS AERUGINOSA, IN THE PRESENCE OF HUMAN SERUM

(71) Applicant: CONTRAFECT CORPORATION, Yonkers, NY (US)

(72) Inventor: Raymond SCHUCH, Mountain Lakes, NJ (US)

(21) Appl. No.: 17/601,244

(22) PCT Filed: Apr. 3, 2020
(86) PCT No.: PCT/US2020/026681

§ 371 Date: Oct. 4, 2021

Related U.S. Application Data
(60) Provisional application No. 62/830,207, filed on Apr. 5, 2019.

(51) Int. Cl. A61K 38/47; A61K 31/407; A61P 31/04

(52) U.S. Cl. CPC A61K 38/47; A61P 31/04; A61K 31/407; C12Y 302/01017

(57) ABSTRACT

Disclosed are novel lysin polypeptides active against Gram-negative bacteria, particularly P. aeruginosa, pharmaceutical compositions containing them and methods for their use to treat Gram-negative bacterial infections and more generally to inhibit the growth, or reduce the population, or kill Gram-negative bacteria, including without limitation disrupting biofilms formed by such bacteria. Certain of the disclosed lysins have been modified in amino acid sequence compared to that of lysins by replacement of certain charged amino acids with noncharged amino acids and/or by fusion at the N- or C-terminus with antibacterial peptide sequences with or without an intervening linker.

### CROSS-REFERENCE TO RELATED APPLICATIONS

This application claims the benefit of, and relies on the filing date of, U.S. provisional patent application No. 62/830,207, filed 5 Apr. 2019, the entire disclosure of which is incorporated herein by reference.

### SEQUENCE LISTING

The instant application contains a Sequence Listing which has been submitted electronically in ASCII format and is hereby incorporated by reference in its entirety. Said ASCII copy, created on 2 Apr. 2020, is named 0341.0025-PCT_ST25.txt and is 62,107 bytes in size.

### BACKGROUND

Gram-negative bacteria, in particular members of the genus Pseudomonas, are an important cause of serious and potentially life-threatening invasive infections. Pseudomonas infection presents a major problem in burn wounds, chronic wounds, chronic obstructive pulmonary disorder (COPD) and other structural lung diseases, cystic fibrosis, surface growth on implanted biomaterials, and within hospital surface and water supplies where it poses a host of threats to vulnerable patients, such as immunosuppressed patients and patients in intensive care (ICU).

Once established in the patient, P. aeruginosa can be especially difficult to treat. The genome encodes a host of resistance genes, including multidrug efflux pumps and enzymes conferring resistance to beta-lactam and aminoglycoside antibiotics, making therapy against this Gram-negative pathogen particularly challenging due to the lack of novel antimicrobial therapeutics. This challenge is compounded by the ability of P. aeruginosa to grow in a biofilm, which may enhance its ability to cause infections by protecting bacteria from host defenses and conventional antimicrobial chemotherapy.

In the healthcare setting, the incidence of drug resistant strains of Pseudomonas aeruginosa is increasing. A multistate point-prevalence survey estimated that P. aeruginosa caused 7% of all healthcare-acquired infections (HAIs) (1). More than 6,000 (13%) of the 51,000 HAIs caused by P. aeruginosa annually are multi-drug resistant (MDR), with roughly 400 deaths per year (2). Extensively drug resistant (XDR) and pan-drug-resistant (PDR) strains represent emerging threats for which there are limited or no available treatments (3). Invasive P. aeruginosa infections including bloodstream infections (BSIs), which are among the most lethal HAIs—for example, P. aeruginosa accounts for 3 to 7% of all BSIs, with mortality rates between 27 and 48% (4). The incidence of invasive bloodstream infections including those caused by P. aeruginosa may be underestimated since the majority of healthcare in the USA is performed in smaller, non-teaching community hospitals. In an observational study of BSIs in community hospitals P. aeruginosa was one of the top 4 MDR pathogens (5) and overall hospital mortality was 18%. Additionally, outbreaks of MDR P. aeruginosa are well described (6). Poor outcomes are associated with MDR stains of P. aeruginosa that frequently require treatment with drugs of last resort such as colistin (7). There is clearly an unmet medical need for different antimicrobials with novel mechanisms to target MDR P. aeruginosa for the treatment of invasive infections including but not limited to BSIs.

An innovative approach to treating bacterial infections focuses on a family of bacteriophage-encoded cell wall peptidoglycan (PG) hydrolases called lysins (8). Lysin technology is currently based on the use of purified recombinant lysin proteins that act externally on a range of Gram-positive (GP) pathogens, resulting in lysis of the bacterial cell on contact with multi-log-fold killing. Lysins act as “molecular scissors” to degrade the peptidoglycan (PG) meshwork responsible for maintaining cell shape and for withstanding the internal osmotic pressure. Degradation of PG results in osmotic lysis. In addition to rapid kill and a novel mode of action compared to antibiotics, other hallmarks of lytic activity include anti-biofilm activity, absence of pre-existing resistance, potent synergy with antibiotics (in sub-minimum inhibitory concentrations (MIC)), and the suppression of resistance to antibiotics when antibiotics are used in addition to lysins. Importantly, multiple researcher groups have demonstrated the ability of topical, intra-nasal, and parenteral dosing with lysins to control antibiotic resistant GP bacterial pathogens in multiple animal models (9-11).

Lysin technology was originally developed to treat GP pathogens. The development of lysins to target Gram-negative (GN) bacteria has heretofore been limited. The outer membrane (OM) of Gram-negative bacteria plays a critical role as a barrier to extracellular macromolecules and limits access to subjacent peptidoglycan (12-14).

The OM is the distinguishing feature of GN bacteria and comprises a lipid bilayer with an internal leaflet of phospholipids and an external amphiphilic leaflet largely consisting of lipopolysaccharide (LPS) (15). The LPS has three main sections: a hexa-acylated glucosamine-based phospholipid called lipid A, a polysaccharide core and an extended, external polysaccharide chain called O-antigen. The OM presents a non-fluid continuum stabilized by three major interactions, including: i) the avid binding of LPS molecules to each other, especially if cations are present to neutralize phosphate groups; ii) the tight packing of largely saturated acyl chains; and iii) hydrophobic stacking of the lipid A moiety. The resulting structure is a barrier for both hydrophobic and hydrophilic molecules. Below the OM, the PG forms a thin layer that is very sensitive to hydrolytic cleavage—unlike the PG of GP bacteria which is 30-100 nm thick and consists of up to 40 layers, the PG of GN bacteria is only 2-3 nm thick and consists of only 1-3 layers. Potent antimicrobial activity could be achieved if lysins targeting GN bacteria are engineered to penetrate the OM either alone or in combination with OM-destabilizing agents and/or antibiotics.

Accordingly, the discovery and development of GN lysins that penetrate the OM is an important goal and would fulfill an important yet unmet need to devise effective therapies for treating or preventing Gram-negative bacterial infections. Multiple agents with OM-permeabilizing and OM-disrupting activities have been previously described. For example, poly-cationic compounds, including polymyxin antibiotics and aminoglycosides, compete with stabilizing divalent cations in the OM for interactions with phospholipids in LPS, leading to disorganization of the OM (16). Similarly, EDTA and weak acids chelate the divalent cations leading to OM disorganization (17). A large group of naturally occurring antimicrobial peptides and synthetic peptidomimetics thereof (herein referred to as AMPs) are also known to penetrate the OM based on a self-promoted uptake pathway (18-20). Translocation of both poly-cationic and amphipathic AMPs is driven by a primary electrostatic interaction with the LPS, followed by cation displacement, membrane disorganization and transient openings, and in some cases internalization of the AMP. The membrane-interacting antimicrobial activity of many AMPs, can be “activated” in blood by strategically engineering the amphipathic domains either by altering hydrophobicity, total charge, and the positioning of polar residues in the hydrophobic face or by incorporating D,L residues in place of all-L counterparts (18, 19, 21, 22).

Lysin technology has been advanced to address GN pathogens using a variety of techniques to enable OM penetration, as outlined herein. In this respect, previously-filed an International patent Application, PCT/US2016/052338 filed Sep. 16, 2016 and published as WO/2017/049233 is fully incorporated by reference herein for all purposes. For example, lysins GN2, GN4, GN14, GN43, and GN37 were first disclosed in the foregoing PCT Application.

Recent studies identified lysins with intrinsic antimicrobial activity against GN bacteria (12, 13, 17). The antimicrobial effect in several cases is attributed to N- or C-terminal amphipathic or poly-cationic α-helical domains that drive penetration of the LPS and translocation across the OM, resulting in PG degradation and osmotic lysis. Interestingly, access of such lysins to the PG can be facilitated by OM-destabilizing compounds including EDTA and mild organic acids. Although combinations with EDTA and mild organic acids are not practical as drugs, the findings illustrate the concept of facilitating GN lytic activity.

A more recent approach uses GN lysins fused to specific α-helical domains with polycationic, amphipathic, and hydrophobic features to promote translocation across the OM. These findings have resulted in GN lysins called “artilysins”, which are highly active in vitro and are envisioned for topical applications (17). However, low activity has been reported for artilysins in vivo. Consistently, artilysin GN126 listed as a control in the present disclosure (see Table 4) also exhibited low activity.

Despite the in vitro potency of artilysins and lysins, including GN lysins, with intrinsic antimicrobial activity, a major limitation remains with respect to a distinct lack of activity in human blood matrices, making systemic therapy a challenge (13, 14). It is believed that physiologic salt and divalent cations compete for LPS binding sites and interfere with the α-helical translocation domains of lysins, including GN lysins, thereby restricting activity in blood and more specifically in the presence of serum, therefore limiting the possibility to use lysins for treating invasive infections (23). A similar lack of activity in blood has been reported for multiple different OM-penetrating and destabilizing AMPs (18-20, 22).

### SUMMARY

A major design challenge facing GN lysin development for the treatment of invasive infections via systemic administration is the need to alleviate the inactivation in blood (or for example in human serum).

Native GN lysins with intrinsic activity (i.e., high-level activity in HEPES buffer and low-level activity in human serum) were first identified and then modified by replacement of charged amino acids with non-charged ones and/or fusion with an alpha-helical antimicrobial peptide for improved activity and improved activity in serum.

Based on this work, putative native lysins were identified and were evaluated for activity. The lysins are listed in Table 3 and described by their sequences. The unmodified lysins exhibit varying levels of activity in the presence of human serum.

Modifications of the lysin proteins were based on the following: i) incorporation of amino acid substitutions into the lysin protein to change the overall pI of the molecule to facilitate OM penetration or reduce sensitivity to human serum or both; and/or ii) the fusion of an antimicrobial peptide sequence (preferably, one known to be active in serum) to the N- or C-terminus of the lysin to form a fusion polypeptide to facilitate outer membrane penetration and translocation.

Modified GN-lysins were obtained by modifying lysin proteins as described herein. The modified GN-lysins are demonstrated to exhibit improved activity in human serum compared to that of the parent (unmodified) lysins. Charged amino acid residues of native lysin proteins were mutagenized randomly by noncharged amino acid residues and the resulting polypeptides were tested for activity, including activity in the presence of human serum. The active modified polypeptides typically differed from the parent polypeptides in 1 to 3 amino acid residues. Alternatively, or additionally, antimicrobial peptide (AMP) sequences were fused onto the native or modified GN lysin sequences with or without a linker. The antimicrobial peptides are characterized by an alpha-helical domain to mediate outer membrane disruption and translocation of the lysin. The linkers are short peptide sequences 5 to 20 amino acids in length which are flexible (for example are rich in serine and/or glycine residues) and are designed not to perturb the structure of either the AMP or the lysin portion of the fusion polypeptide and to allow each to move freely.

Each of the putative lysins and modified GN-lysins described herein, have been or can be purified to >90% homogeneity and examined in a series of assays to assess in vitro activity.

The present disclosure encompasses lysin polypeptides and modified lysin polypeptides which are synthetically and/or recombinantly produced. Disclosed herein are novel lysin polypeptides and modified lysin polypeptides, as well as the use of said polypeptides for the treatment of infections with Gram-negative bacteria and, especially in the presence of blood matrices, e.g., human serum.

What is more, disclosed herein is the use of lysin polypeptides and modified lysin polypeptides for disrupting biofilms comprising Gram-negative microorganisms, for example in prosthetic or in other medical devices, in vivo, ex vivo or in vitro. The Gram-negative microorganisms of biofilms include Pseudomonas species, for example, Pseudomonas aeruginosa.

In one aspect, the present disclosure is directed to a pharmaceutical composition or drug formulation comprising an effective amount of an isolated lysin polypeptide having an amino acid sequence selected from the group consisting of SEQ ID NO: 2, 4, 5-9 and SEQ ID NO: 13-27 or a peptide having at least 80% sequence identity therewith, said peptide having lytic activity, wherein the lysin polypeptide inhibits the growth, or reduces the population, or kills at least one species of Gram-negative bacteria; and a pharmaceutically acceptable carrier.

In an embodiment the pharmaceutical composition comprises an effective amount of at least one lysin polypeptide selected from the group consisting of peptides GN3, CN147, GN146, GN156, GN54, GN92, GN121, GN94, GN9, GN10, GN13, GN17, GN105, GN108, GN123, GN150, GN200, GN201, GN203, GN204 and GN205 or a fragment thereof maintaining lytic activity, wherein the lysin polypeptide or fragment inhibits the growth, or reduces the population, or at least one species of Gram-negative bacteria; and a pharmaceutically acceptable carrier.

The present pharmaceutical compositions/drug formulations, in one embodiment, comprise an effective amount of at least one lysin polypeptide and at least one antibiotic suitable for the treatment of Gram-negative bacteria. In some embodiments, the composition is a combination of two components to be administered combinedly or separately, one containing a lysin in accordance with the present disclosure and one containing an antibiotic. In some embodiments, the antibiotic is provided in a suboptimal dose. In some embodiments the antibiotic may be one to which the Gram-negative bacteria have developed resistance, the use of the lysin serving to overcome this resistance).

In some embodiments, the present compositions (with or without antibiotic) or combinations (with lysin and antibiotic) are adapted for oral, topical, parenteral or inhalable administration. In some embodiments, one component of the combination may be adapted to be administered by a different route than the other component. For example, in the experiments detailed below, antibiotics are administered subcutaneously (SC) whereas the lysins are administered intravenously (IV).

In an embodiment, the antibiotic may be selected from the list of GN suitable antibiotics provided below and combinations thereof. In a more specific embodiment, the antibiotic may be selected from amikacin, azithromycin, aztreonam, ciprofloxacin, colistin, rifampicin, carbapenems and tobramycin and combinations of two or more of the foregoing.

Certain embodiments of the present disclosure contemplate a sterile container that contains one of the above-mentioned pharmaceutical compositions comprising a lysin polypeptide and optionally one or more additional components. By way of example, but not limitation, the sterile container is one component of a kit; the kit may also contain, for example, a second sterile container that contains at least one additional therapeutic agent. Thus, one of the combinations of GN antibiotic and GN lysin disclosed herein may optionally be provided in such a kit.

In an aspect, disclosed herein is a vector comprising a nucleic acid molecule which encodes a lysin peptide having an amino acid sequence selected from the group consisting of SEQ ID NO: 2, 4, 5-9 and SEQ ID NO: 13-27, or a peptide having at least 80% sequence identity therewith, said peptide having lytic activity, wherein the encoded lysin polypeptide inhibits the growth, or reduces the population, or kills at least one species of Gram-negative bacteria in the absence or presence of human serum.

In another embodiment, the vector is a recombinant expression vector comprising a nucleic acid encoding one of the foregoing lysin polypeptides including the at least 80% sequence identity variants thereof, wherein the encoded lysin peptide has the property of inhibiting the growth, or reducing the population, or at least one species of Gram-negative bacteria in the absence and/or presence of human serum, the nucleic acid being operatively linked to a heterologous promoter.

A host cell comprising the foregoing vectors are also contemplated. In some embodiments the nucleic acid sequence is a cDNA sequence.

In yet another aspect, the disclosure is directed to isolated, purified nucleic acid encoding a lysin polypeptide comprising a sequence selected from the group consisting of SEQ ID NO: 2, 4, 5-9 and SEQ ID NO: 13-27. In an alternative embodiment, the isolated, purified nucleic acid comprises a nucleotide sequence selected from the group consisting of SEQ ID NO: 33 through SEQ ID NO: 54, degenerate code thereof, and transcripts thereof. In accordance with the embodiments presented herein, a defined nucleic acid includes not only the identical nucleic acid but also any minor base variations including, in particular, substitutions resulting in a synonymous codon (a different codon specifying the same amino acid residue). The claims drawn to nucleic acid will thus be deemed to encompass the complementary sequence to any recited single-stranded sequence. Optionally, the nucleic acid is cDNA.

In another aspect, the present disclosure is directed to a method for resensitizing a Gram-negative bacterium to an antibiotic, which method comprises contacting the Gram-negative bacterium with the antibiotic and a lysin as described herein, thereby resensitizing the Gram-negative bacterium to the antibiotic. Typically, the lysin and the antibiotic exhibit synergystic activity against the gram negative bacterium.

In other aspects, the present disclosure is directed to various methods/uses. One such is a method/use for inhibiting the growth, or reducing the population, or killing of at least one species of Gram-negative bacteria, the method comprising contacting the bacteria with a composition comprising an effective amount of a GN lysin polypeptide comprising a sequence selected from the group consisting of SEQ ID NO: 2, 4, 5-9 and SEQ ID NO: 13-27, or a peptide having at least 80% sequence identity therewith, said peptide having lytic activity for a period of time sufficient to inhibit said growth or reduce said population or kill said at least one species of Gram-negative bacteria in the absence and/or presence of human serum.

Another such method/use is for inhibiting the growth, or reducing the population, or killing of at least one species of Gram-negative bacteria, the method comprising contacting the bacteria with a composition comprising an effective amount of at least one GN lysin polypeptide selected from the group consisting of the GN lysins as described in SEQ ID NO: 2, 4, 5-9 and SEQ ID NO: 13-27, or active fragments thereof, wherein the polypeptide or active fragment has the property of inhibiting the growth, or reducing the population, or killing P. aeruginosa and optionally at least one other species of Gram-negative bacteria in the absence and/or presence of human serum.

Another method/medical use is for treating a bacterial infection caused by a Gram-negative bacterium, such as P. aeruginosa or A. baumannii, including antibiotic-resistant Gram-negative bacterium, such as carbapenem-resistant Gram-negative bacterium including carbapenem-resistant P. aeruginosa or A. baumannii comprising administering to a subject diagnosed with, at risk for, or exhibiting symptoms of a bacterial infection, one or more of the foregoing compositions.

In any of the foregoing methods/medical uses the Gram-negative bacterium is at least one selected from the group consisting of Acinetobacter baumannii, Pseudomonas aeruginosa, E. coli, Klebsiella pneumoniae, Enterobacter cloacae, Salmonella spp., N. gonorrhoeae, and Shigella spp. Alternatively, the Gram-negative bacteria is Pseudomonas aeruginosa.

Another method/medical use is for treating or preventing a topical or systemic pathogenic bacterial infection caused by a Gram-negative bacteria comprising administering to a subject in need of treatment one of the foregoing compositions. Topical infections include infections that can be treated by local or topical application of an antibacterial agent. Examples of topical infections include those confined to a particular location, such as an organ or tissue or an implanted prosthesis or other medical device. Examples are infections of the skin, gums, infected wounds, infections of the ear etc., infections in the area where a catheter is installed etc.

Another such method/medical use is for preventing or treating a bacterial infection comprising co-administering to a subject diagnosed with, at risk for, or exhibiting symptoms of a bacterial infection, a combination of a first effective amount of one of the foregoing compositions and a second effective amount of an antibiotic suitable for the treatment of Gram-negative bacterial infection.

### DETAILED DESCRIPTION

### Definitions

As used herein, the following terms and cognates thereof shall have the meanings ascribed to them below unless the context clearly indicates otherwise.

“Carrier” refers to a solvent, additive, excipient, dispersion medium, solubilizing agent, coating, preservative, isotonic and absorption delaying agent, surfactant, propellant, diluent, vehicle and the like with which an active compound is administered. Such carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil, and the like.

“Pharmaceutically acceptable carrier” refers to any and all solvents, additives, excipients, dispersion media, solubilizing agents, coatings, preservatives, isotonic and absorption delaying agents, surfactants, propellants, diluents, vehicles and the like that are physiologically compatible. The carrier(s) must be “acceptable” in the sense of not being deleterious to the subject to be treated in amounts typically used in medicaments. Pharmaceutically acceptable carriers are compatible with the other ingredients of the composition without rendering the composition unsuitable for its intended purpose. Furthermore, pharmaceutically acceptable carriers are suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers or excipients include any of the standard pharmaceutical carriers such as phosphate buffered saline solutions, water, and emulsions such as oil/water emulsions and microemulsions. Suitable pharmaceutical carriers are described, for example, in Remington's Pharmaceutical Sciences by E. W. Martin, 18th Edition. The pharmaceutically acceptable carrier may be a carrier that does not exist in nature.

“Bactericidal” or “bactericidal activity” refers to the property of causing the death of bacteria or capable of killing bacteria to an extent of at least a 3-log 10 (99.9%) or better reduction among an initial population of bacteria over an 18-24 hour period.

“Bacteriostatic” or “bacteriostatic activity” refers to the property of inhibiting bacterial growth, including inhibiting growing bacterial cells, thus causing a 2-log 10 (99%) or better and up to just under a 3-log reduction among an initial population of bacteria over an 18-24 hour period.

“Antibacterial” refers to both bacteriostatic and bactericidal agents.

“Antibiotic” refers to a compound having properties that have a negative effect on bacteria, such as lethality or reduction of growth. An antibiotic can have a negative effect on Gram-positive bacteria, Gram-negative bacteria, or both. By way of example, an antibiotic can affect cell wall peptidoglycan biosynthesis, cell membrane integrity, or DNA or protein synthesis in bacteria. Nonlimiting examples of antibiotics active against Gram-negative bacteria include cephalosporins, such as ceftriaxone-cefotaxime, ceftazidime, cefepime, cefoperazone, and ceftobiprole; fluoroquinolones such as ciprofloxacin and levofloxacin; aminoglycosides such as gentamicin, tobramycin, and amikacin; piperacillin, ticarcillin, imipenem, meropenem, doripenem, broad spectrum penicillins with or without beta-lactamase inhibitors, rifampicin, polymyxin B, and colistin.

“Drug resistant” generally refers to a bacterium that is resistant to the antibacterial activity of a drug. When used in certain ways, drug resistance may specifically refer to antibiotic resistance. In some cases, a bacterium that is generally susceptible to a particular antibiotic can develop resistance to the antibiotic, thereby becoming a drug resistant microbe or strain. A “multi-drug resistant” (“MDR”) pathogen is one that has developed resistance to at least two classes of antimicrobial drugs, each used as monotherapy. For example, certain strains of S. aureus have been found to be resistant to several antibiotics including methicillin and/or vancomycin (Antibiotic Resistant Threats in the United States, 2013, U.S. Department of Health and Services, Centers for Disease Control and Prevention). One skilled in the art can readily determine if a bacterium is drug resistant using routine laboratory techniques that determine the susceptibility or resistance of a bacterium to a drug or antibiotic.

“Effective amount” refers to an amount which, when applied or administered in an appropriate frequency or dosing regimen, is sufficient to prevent, reduce, inhibit, or eliminate bacterial growth or bacterial burden or to prevent, reduce, or ameliorate the onset, severity, duration, or progression of the disorder being treated (for example, Gram-negative bacterial pathogen growth or infection), prevent the advancement of the disorder being treated, cause the regression of the disorder being treated, or enhance or improve the prophylactic or therapeutic effect(s) of another therapy, such as antibiotic or bacteriostatic therapy.

“Co-administer” refers to the administration of two agents, such as a lysin or lysin-AMP polypeptide and an antibiotic or any other antibacterial agent, in a sequential manner, as well as administration of these agents in a substantially simultaneous manner, such as in a single mixture/composition or in doses given separately, but nonetheless administered substantially simultaneously to the subject, for example at different times in the same day or 24-hour period. Such co-administration of two agents, such as a lysin or lysin-AMP polypeptide with one or more additional antibacterial agents can be provided as a continuous treatment lasting up to days, weeks, or months. Additionally, depending on the use, the co-administration need not be continuous or coextensive. For example, if the use were as a topical antibacterial agent to treat, e.g., a bacterial ulcer or an infected diabetic ulcer, a lysin or lysin-AMP polypeptide could be administered only initially within 24 hours of an additional antibiotic, and then the additional antibiotic use may continue without further administration of the lysin or lysin-AMP polypeptide.

“Subject” refers to a mammal, a plant, a lower animal, a single cell organism, or a cell culture. For example, the term “subject” is intended to include organisms, e.g., prokaryotes and eukaryotes, which are susceptible to or afflicted with bacterial infections, for example Gram-positive or Gram-negative bacterial infections. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or susceptible to infection by Gram-negative bacteria, whether such infection be systemic, topical or otherwise concentrated or confined to a particular organ or tissue.

“Polypeptide” is used herein interchangeably with the term “peptide” or “protein” and refers to a polymer made from amino acid residues and generally having at least about 30 amino acid residues. The term includes not only polypeptides in isolated form, but also active fragments and derivatives thereof. The term “polypeptide” also encompasses fusion proteins or fusion polypeptides comprising a lysin or AMP as described herein and maintaining, for example a lytic function. Depending on context, a polypeptide can be a naturally occurring polypeptide or a recombinant, engineered, or synthetically produced polypeptide. A particular lysin polypeptide, for example, can be, for example, derived or removed from a native protein by enzymatic or chemical cleavage, or can be prepared using conventional peptide synthesis techniques (e.g., solid phase synthesis) or molecular biology techniques (such as those disclosed in Sambrook, J. et al., Molecular Cloning: A Laboratory Manual, Cold Spring Harbor Press, Cold Spring Harbor, N.Y. (1989)) or can be strategically truncated or segmented yielding active fragments, maintaining, e.g., lytic activity against the same or at least one common target bacterium.

“Fusion polypeptide” refers to an expression product resulting from the fusion of two or more nucleic acid segments, resulting in a fused expression product typically having two or more domains or segments, which typically have different properties or functionality. In a more particular sense, the term “fusion polypeptide” may also refer to a polypeptide or peptide comprising two or more heterologous polypeptides or peptides covalently linked, either directly or via an amino acid or peptide linker. The polypeptides forming the fusion polypeptide are typically linked C-terminus to N-terminus, although they can also be linked C-terminus to C-terminus, N-terminus to N-terminus, or N-terminus to C-terminus. The term “fusion polypeptide” can be used interchangeably with the term “fusion protein.” The open-ended expression “a polypeptide comprising” a certain structure includes larger molecules than the recited structure, such as fusion polypeptides.

“Heterologous” refers to nucleotide, peptide, or polypeptide sequences that are not naturally contiguous. For example, in the context of the present disclosure, the term “heterologous” can be used to describe a combination or fusion of two or more peptides and/or polypeptides wherein the fusion peptide or polypeptide is not normally found in nature, such as for example a lysin or active fragment thereof and an antimicrobial peptide, including a cationic and/or a polycationic peptide, an amphipathic peptide, a sushi peptide (Ding et al. Cell Mol Life Sci., 65(7-8):1202-19 (2008)), a defensin peptide (Ganz, T. Nature Reviews Immunology 3, 710-720 (2003)), a hydrophobic peptide, which may have enhanced lytic activity.

“Active fragment” refers to a portion of a polypeptide that retains one or more functions or biological activities of the isolated polypeptide from which the fragment was taken, for example bactericidal activity against one or more Gram-negative bacteria.

“Amphipathic peptide” refers to a peptide having both hydrophilic and hydrophobic functional groups. In certain embodiments, secondary structure may place hydrophobic and hydrophilic amino acid residues at opposite sides (e.g., inner side vs outer side when the peptide is in a solvent, such as water) of an amphipathic peptide. These peptides may in certain embodiments adopt a helical secondary structure, such as an alpha-helical secondary structure.

“Cationic peptide” refers to a peptide having a high percentage of positively charged amino acid residues. In certain embodiments, a cationic peptide has a pKa-value of 8.0 or greater. The term “cationic peptide” in the context of the present disclosure also encompasses polycationic peptides that are synthetically produced peptides composed of mostly positively charged amino acid residues, such as lysine (Lys) and/or arginine (Arg) residues. The amino acid residues that are not positively charged can be neutrally charged amino acid residues, negatively charged amino acid residues, and/or hydrophobic amino acid residues.

“Hydrophobic group” refers to a chemical group such as an amino acid side chain that has low or no affinity for water molecules but higher affinity for oil molecules. Hydrophobic substances tend to have low or no solubility in water or aqueous phases and are typically apolar but tend to have higher solubility in oil phases. Examples of hydrophobic amino acids include glycine (Gly), alanine (Ala), valine (Val), Leucine (Leu), isoleucine (Ile), proline (Pro), phenylalanine (Phe), methionine (Met), and tryptophan (Trp).

“Augmenting” refers to a degree of activity of an agent, such as antimicrobial activity, that is higher than it would be otherwise. “Augmenting” encompasses additive as well as synergistic (superadditive) effects.

“Synergistic” or “superadditive” refers to a beneficial effect brought about by two substances in combination that exceeds the sum of the effects of the two agents working independently. In certain embodiments the synergistic or superadditive effect significantly, i.e., statistically significantly, exceeds the sum of the effects of the two agents working independently. One or both active ingredients may be employed at a sub-threshold level, i.e., a level at which if the active substance is employed individually produces no or a very limited effect. The effect can be measured by assays such as the checkerboard assay, described here.

“Treatment” refers to any process, action, application, therapy, or the like, wherein a subject, such as a human being, is subjected to medical aid with the object of curing a disorder, eradicating a pathogen, or improving the subject's condition, directly or indirectly. Treatment also refers to reducing incidence, alleviating symptoms, eliminating recurrence, preventing recurrence, preventing incidence, reducing the risk of incidence, improving symptoms, improving prognosis, or combinations thereof. “Treatment” may further encompass reducing the population, growth rate, or virulence of a bacteria in the subject and thereby controlling or reducing a bacterial infection in a subject or bacterial contamination of an organ, tissue, or environment. Thus “treatment” that reduces incidence may, for example, be effective to inhibit growth of at least one Gram-negative bacterium in a particular milieu, whether it be a subject or an environment. On the other hand, “treatment” of an already established infection refers to inhibiting the growth, reducing the population, killing, including eradicating, a Gram-negative bacteria responsible for an infection or contamination.

“Preventing” refers to the prevention of the incidence, recurrence, spread, onset or establishment of a disorder such as a bacterial infection. It is not intended that the present disclosure be limited to complete prevention or to prevention of establishment of an infection. In some embodiments, the onset is delayed, or the severity of a subsequently contracted disease or the chance of contracting the disease is reduced, and such constitute examples of prevention.

“Contracted diseases” refers to diseases manifesting with clinical or subclinical symptoms, such as the detection of fever, sepsis, or bacteremia, as well as diseases that may be detected by growth of a bacterial pathogen (e.g., in culture) when symptoms associated with such pathology are not yet manifest.

The term “derivative” in the context of a peptide or polypeptide or active fragments thereof is intended to encompass, for example, a polypeptide modified to contain one or more chemical moieties other than an amino acid that do not substantially adversely impact or destroy the polypeptide's activity (e.g., lytic activity). The chemical moiety can be linked covalently to the peptide, e.g., via an amino terminal amino acid residue, a carboxy terminal amino acid residue, or at an internal amino acid residue. Such modifications may be natural or non-natural. In certain embodiments, a non-natural modification may include the addition of a protective or capping group on a reactive moiety, addition of a detectable label, such as antibody and/or fluorescent label, addition or modification of glycosylation, or addition of a bulking group such as PEG (pegylation) and other changes known to those skilled in the art. In certain embodiments, the non-natural modification may be a capping modification, such as N-terminal acetylations and C-terminal amidations. Exemplary protective groups that may be added to lysin polypeptides or AMPs include, but are not limited to, t-Boc and Fmoc. Commonly used fluorescent label proteins such as, but not limited to, green fluorescent protein (GFP), red fluorescent protein (RFP), cyan fluorescent protein (CFP), yellow fluorescent protein (YFP), and mCherry, are compact proteins that can be bound covalently or noncovalently to a polypeptide or fused to a polypeptide without interfering with normal functions of cellular proteins. In certain embodiments, a polynucleotide encoding a fluorescent protein may be inserted upstream or downstream of the lysin or AMP polynucleotide sequence. This will produce a fusion protein (e.g., Lysin Polypeptide::GFP) that does not interfere with cellular function or function of a polypeptide to which it is attached. Polyethylene glycol (PEG) conjugation to proteins has been used as a method for extending the circulating half-life of many pharmaceutical proteins. Thus, in the context of polypeptide derivatives, such as lysin polypeptide derivatives, the term “derivative” encompasses polypeptides, such as lysin polypeptides, chemically modified by covalent attachment of one or more PEG molecules. It is anticipated that lysin polypeptides, such as pegylated lysins, will exhibit prolonged circulation half-life compared to the unpegylated polypeptides, while retaining biological and therapeutic activity.

“Percent amino acid sequence identity” refers to the percentage of amino acid residues in a candidate sequence that are identical with the amino acid residues in the reference polypeptide sequence, such as a lysin polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for example, using publicly available software such as BLAST or software available commercially, for example from DNASTAR. Two or more polypeptide sequences can be anywhere from 0-100% identical, or any integer value there between. In the context of the present disclosure, two polypeptides are “substantially identical” when at least 80% of the amino acid residues (such as at least about 85%, at least about 90%, at least about 92.5%, at least about 95%, at least about 98%, or at least about 99%) are identical. The term “percent (%) amino acid sequence identity” as described herein applies to peptides as well. Thus, the term “substantially identical” will encompass mutated, truncated, fused, or otherwise sequence-modified variants of isolated lysin polypeptides and peptides and AMPs described herein, and active fragments thereof, as well as polypeptides with substantial sequence identity (e.g., at least 80%, at least 85%, at least 90%, at least 92.5%, at least 95%, at least 98%, or at least 99% identity as measured for example by one or more methods referenced above) as compared to the reference (wild type or other intact) polypeptide.

As used herein, two amino acid sequences are “substantially homologous” when at least about 80% of the amino acid residues (such as at least about 85%, at least about 90%, at least about 92.5%, at least about 95%, at least about 98%, or at least about 99%) are identical, or represent conservative substitutions. The sequences of the polypeptides of the present disclosure are substantially homologous when one or more, such as up to 10%, up to 15%, or up to 20% of the amino acids of the polypeptide, such as the lysin, AMP, and/or fusion polypeptides described herein, are substituted with a similar or conservative amino acid substitution, and wherein the resulting peptides have at least one activity (e.g., antibacterial effect) and/or bacterial specificities of the reference polypeptide, such as the lysin, AMP, and/or fusion polypeptides described herein.

As used herein, a “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Inhalable composition” refers to pharmaceutical compositions of the present disclosure that are formulated for direct delivery to the respiratory tract during or in conjunction with routine or assisted respiration (e.g., by intratracheobronchial, pulmonary, and/or nasal administration), including, but not limited to, atomized, nebulized, dry powder, and/or aerosolized formulations.

“Biofilm” refers to bacteria that attach to surfaces and aggregate in a hydrated polymeric matrix that may be comprised of bacterial- and/or host-derived components. A biofilm is an aggregate of microorganisms in which cells adhere to each other on a biotic or abiotic surface. These adherent cells are frequently embedded within a matrix comprised of, but not limited to, extracellular polymeric substance (EPS). Biofilm EPS, which is also referred to as slime (although not everything described as slime is a biofilm) or plaque, is a polymeric conglomeration generally composed of extracellular DNA, proteins, and polysaccharides.

“Suitable” in the context of an antibiotic being suitable for use against certain bacteria refers to an antibiotic that was found to be effective against those bacteria even if resistance subsequently developed.

“Outer Membrane” or “OM” refers to a feature of Gram-negative bacteria. The outer membrane is comprised of a lipid bilayer with an internal leaflet of phospholipids and an external amphiphilic leaflet largely consisting of lipopolysaccharide (LPS). The LPS has three main sections: a hexa-acylated glucosamine-based phospholipid called lipid A, a polysaccharide core and an extended, external polysaccharide chain called 0-antigen. The OM presents a non-fluid continuum stabilized by three major interactions, including: i) the avid binding of LPS molecules to each other, especially if cations are present to neutralize phosphate groups; ii) the tight packing of largely saturated acyl chains; and iii) hydrophobic stacking of the lipid A moiety. The resulting structure is a barrier for both hydrophobic and hydrophilic molecules. Below the OM, the peptidoglycan forms a thin layer that is very sensitive to hydrolytic cleavage—unlike the peptidoglycan of Gram-negative bacteria which is 30-100 nanometers (nm) thick and consists of up to 40 layers, the peptidoglycan of Gram-negative bacteria is only 2-3 nm thick and consists of only 1-3 layers.

Identification of Lysins with Bactericidal Activity Against P. aeruginosa in Human Serum. The present disclosure is based on identification of five lysins with potent antibacterial activity against exponential phase Pseudonionas aeruginosa strain PAOI (Examples 1 and 2). This strain is representative of P. aeruginosa strains. To identify the lysin polypeptides of the present disclosure, a bioinformatics-based approach was used, coupled with an antibacterial screen. Putative lysins and lysin-like molecules (see Table 1) were identified from the GenBank database. The GenBank sequences were annotated as either hypothetical or predicted proteins, and in some cases were listed as putative phage proteins and/or putative lysins). Any reports of activity for these polypeptides is not known.

TABLE 1

| Lysin | pl | GenBank Acssession No. |
|---|---|---|
| GN3 | 9.98 | WP_012273008.1 |
| GN13 | 9.47 | YP_00638255.1 |
| GN17 | 7.85 | ACD38663.1 |
| GN9 | 8.85 | ECJ78460.1 |
| GN10 | 9.70 | YP_002600773.1 |
| GN105 | 9.01 | WP_016046696.1 |
| GN108 | 9.28 | YP_009288673.1 |
| GN123 | 9.30 | YP_009217242.1 |
| GN150 | 9.30 | WP_034684053.1 |
| GN203 | 7.87 | YP_024745.1 |

Identification of Modified Lysins with Improved Bactericidal Activity Against P. aeruginosa in Human Serum. Five lysins, GN3, GN150, GN203, GN4 and GN37, were used to generate 12 novel GN-lysin derivatives. See Table. It is contemplated that the modifications (amino acid substitutions or N- or C-terminal peptide fusion with or without linker) could be individually or simultaneously applied to a native lysin or to a modified lysin. Thus, for example, the addition of an N- and/or C-terminal peptide disclosed in Table 2 is contemplated for modifying lysin polypeptides. As a more specific example, the peptide that is part of GN156 or GN92 is contemplated for GN147 even though such a construct has not been exemplified in Table 2. And such a peptide can be added for example to either GN4 or GN146. In other words, an antimicrobial peptide can be fused to the N- or the C-terminus of a native lysin or a lysin modified by noncharged amino acid substitutions in place of charged amino acid residues. Furthermore, the N-terminal and/or C-terminal peptides and/or antimicrobial peptides may be connected to a lysin polypeptide via a linker domain, for example, a linker domain defined in Table 0.2 or another appropriate linker, as described in this section above.

TABLE 2

| | | Native Lysin (Accession | |
|---|---|---|---|
| Lysin | pI | number/Class)* | Modification |
| GN147 | 9.39 | GN3 | Amino acid substitutions |
| | | (WP_012273008.1/Lysozyme) | (R101D, R117H) |
| GN146 | 8.01 | GN4 | Amino acid substitutions (K99D, |
| | | (YP_002284361.1/Lysozyme) | R115H) |
| GN156 | 10.51 | GN4 | Addition of N-terminal peptide |
| | | (YP_002284361.1/Lysozyme) | (GPRRPRRPGRRAPV; SEQ ID |
| | | | NO: 28) |
| GN92 | 9.93 | GN4 | Addition of N-terminal peptide |
| | | (YP_002284361.1/Lysozyme) | (KFFKFFKFFK; SEQ ID |
| | | | NO: 29) with linker |
| | | | (AGAGAGAGAGAGAGAGAS; |
| | | | SEQ ID NO: 31) |
| GN54 | 10.34 | GN4 | Addition of N-terminal peptide |
| | | (YP_002284361.1/Lysozyme) | (KRKKRKKRK; SEQ ID |
| | | | NO: 30) with linker |
| | | | (AGAGAGAGAGAGAGAGAS; |
| | | | SEQ ID NO: 31) |
| GN201 | 10.47 | GN3 | Addition of C-terminal peptide |
| | | (WP_012273008.1/Lysozyme) | (GPRRPRRPGRRAPV; SEQ ID |
| | | | NO: 28); Amino acid |
| | | | substitutions (R101D, R117H) |
| GN202 | 10.13 | GN4 | Addition of C-terminal peptide |
| | | (YP_002284361.1/Lysozyme) | (GPRRPRRPGRRAPV; SEQ ID |
| | | | NO: 28); Amino acid |
| | | | substitutions (K99D, R115H) |
| GN121 | 10.13 | GN37 | Addition of C-terminal peptide |
| | | (WP_014102102.1/VanY) | (RKKTRKRLKKIGKVLKWI; |
| | | | SEQ ID NO: 32) |
| GN94 | 9.77 | GN37 | Addition of N-terminal peptide |
| | | (WP_014102102.1/VanY) | (KFFKFFKFFK; SEQ ID |
| | | | NO: 29) with linker |
| | | | (AGAGAGAGAGAGAGAGAS; |
| | | | SEQ ID NO: 31) |
| GN200 | 9.97 | GN150 | Addition of C-terminal peptide |
| | | (WP_034684053.1/VanY) | (RKKTRKRLKKIGKVLKWI; |
| | | | SEQ ID NO: 32) |
| GN204 | 9.88 | GN203 | Addition of C-terminal peptide |
| | | (YP_024745.1/VanY) | (RKKTRKRLKKIGKVLKWI; |
| | | | SEQ ID NO: 32) |
| GN205 | 11.02 | GN3 | Addition of N-terminal peptide |
| | | (WP_012273008.1/Lysozyme) | (GPRRPRRPGRRAPV; SEQ ID |
| | | | NO: 28) |

The present lysins and modified GN-lysins and their amino acid sequences are summarized in Table 3. Also included in Table 3 are unmodified lysins disclosed in WO/2017/049233, as stated above.

TABLE 3

| Lysin | Amino Acid Sequence |
|---|---|
| GN2 | MKISLEGLSLIKKFEGCKLEAYKCSAGVWTIGYGHTAGVKEGDVCTQEEA |
| | EKLLRGDIFKFEEYVQDSVKVDLDQSQFDALVAWTFNLGPGNLRSSTMLK |
| | KLNNGEYESVPFEMRRWNKAGGKTLDGLIRRRQAESLLFESKEWHQV |
| | (SEQ ID NO: 1) |
| GN3 | MRTSQRGLSLIKSFEGLRLQAYQDSVGVWTIGYGTTRGVKAGMKISKDQA |
| | ERMLLNDVQRFEPEVERLIKVPLNQDQWDALMSFTYNLGAANLESSTLRR |
| | LLNAGNYAAAAEQFPRWNKAGGQVLAGLTRRRAAERELFLGAA (SEQ ID |
| | NO: 2) |
| GN4 | MRTSQRGIDLIKSFEGLRLSAYQDSVGVWTIGYGTTRGVTRYMTITVEQAE |
| | RMLSNDIQRFEPELDRLAKVPLNQNQWDALMSFVYNLGAANLASSTLLKL |
| | LNKGDYQGAADQFPRWVNAGGKRLDGLVKRRAAERALFLEPLS (SEQ ID |
| | NO: 3) |
| GN146 | MRTSQRGIDLIKSFEGLRLSAYQDSVGVWTIGYGTTRGVTRYMTITVEQAE |
| | RMLSNDIQRFEPELDRLAKVPLNQNQWDALMSFVYNLGAANLASSTLLDL |
| | LNKGDYQGAADQFPHWVNAGGKRLDGLVKRRAAERALFLEPLS (SEQ ID |
| | NO: 4) |
| GN147 | MRTSQRGLSLIKSFEGLRLQAYQDSVGVWTIGYGTTRGVKAGMKISKDQA |
| | ERMLLNDVQRFEPEVERLIKVPLNQDQWDALMSFTYNLGAANLESSTLRD |
| | LLNAGNYAAAAEQFPHWNKAGGQVLAGLTRRRAAERELFLGAA (SEQ ID |
| | NO: 5) |
| GN156 | GPRRPRRPGRRAPVMRTSQRGIDLIKSFEGLRLSAYQDSVGVWTIGYGTTR |
| | GVTRYMTITVEQAERMLSNDIQRFEPELDRLAKVPLNQNQWDALMSFVYN |
| | LGAANLASSTLLKLLNKGDYQGAADQFPRWVNAGGKRLDGLVKRRAAER |
| | ALFLEPLS (SEQ ID NO: 6) |
| GN92 | KFFKFFKFFKAGAGAGAGAGAGAGAGASMRTSQRGIDLIKSFEGLRLSAY |
| | QDSVGVWTIGYGTTRGVTRYMTITVEQAERMLSNDIQRFEPELDRLAKVPL |
| | NQNQWDALMSFVYNLGAANLASSTLLKLLNKGDYQGAADQFPRWVNAG |
| | GKRLDGLVKRRAAERALFLEPLS (SEQ ID NO: 7) |
| GN54 | KRKKRKKRKAGAGAGAGAGAGAGAGASMRTSQRGIDLIKSFEGLRLSAY |
| | QDSVGVWTIGYGTTRGVTRYMTITVEQAERMLSNDIQRFEPELDRLAKVPL |
| | NQNQWDALMSFVYNLGAANLASSTLLKLLNKGDYQGAADQFPRWVNAG |
| | GKRLDGLVKRRAAERALFLEPLS (SEQ ID NO: 8) |
| GN202 | MRTSQRGIDLIKSFEGLRLSAYQDSVGVWTIGYGTTRGVTRYMTITVEQAE |
| | RMLSNDIQRFEPELDRLAKVPLNQNQWDALMSFVYNLGAANLASSTLLDL |
| | LNKGDYQGAADQFPHWVNAGGKRLDGLVKRRAAERALFLEPLSGPRRPR |
| | RPGRRAPV (SEQ ID NO: 9) |
| GN14 | MNNELPWVAEARKYIGLREDTSKTSHNPKLLAMLDRMGEFSNESRAWWH |
| | DDETPWCGLFVGYCLGVAGRYVVREWYRARAWEAPQLTKLDRPAYGAL |
| | VTFTRSGGGHVGFIVGKDARGNLMVLGGNQSNAVSIAPFAVSRVTGYFWP |
| | SFWRNKTAVKSVPFEERYSLPLLKSNGELSTNEA (SEQ ID NO: 10) |
| GN43 | MKRTTLNLELESNTDRLLQEKDDLLPQSVTNSSDEGTPFAQVEGASDDNTA |
| | EQDSDKPGASVADADTKPVDPEWKTITVASGDTLSTVFTKAGLSTSAMHD |
| | MLTSSKDAKRFTHLKVGQEVKLKLDPKGELQALRVKQSELETIGLDKTDK |
| | GYSFKREKAQIDLHTAYAHGRITSSLFVAGRNAGLPYNLVTSLSNIFGYDID |
| | FALDLREGDEFDVIYEQHKVNGKQVATGNILAARFVNRGKTYTAVRYTNK |
| | QGNTSYYRADGSSMRKAFIRTPVDFARISSRFSLGRRHPILNKIRAHKGVDY |
| | AAPIGTPIKATGDGKILEAGRKGGYGNAVVIQHGQRYRTIYGHMSRFAKGI |
| | RAGTSVKQGQIIGYVGMTGLATGPHLHYEFQINGRHVDPLSAKLPMADPL |
| | GGADRKRFMAQTQPMIARMDQEKKTLLALNKQR (SEQ ID NO: 11) |
| GN37 | MTYTLSKRSLDNLKGVHPDLVAVVHRAIQLTPVDFAVIEGLRSVSRQKELV |
| | AAGASKTMNSRHLTGHAVDLAAYVNGIRWDWPLYDAIAVAVKAAAKEL |
| | GVAIVWGGDWTTFKDGPHFELDRSKYR (SEQ ID NO: 12) |
| GN121 | MTYTLSKRSLDNLKGVHPDLVAVVHRAIQLTPVDFAVIEGLRSVSRQKEL |
| | VAAGASKTMNSRHLTGHAVDLAAYVNGIRWDWPLYDAIAVAVKAAAKE |
| | LGVAIVWGGDWTTFKDGPHFELDRSKYRRKKTRKRLKKIGKVLKWI (SEQ |
| | ID NO: 13) |
| GN94 | KFFKFFKFFKAGAGAGAGAGAGAGAGASMTYTLSKRSLDNLKGVHPDL |
| | VAVVHRAIQLTPVDFAVIEGLRSVSRQKELVAAGASKTMNSRHLTGHAVD |
| | LAAYVNGIRWDWPLYDAIAVAVKAAAKELGVAIVWGGDWTTFKDGPHF |
| | ELDRSKYR (SEQ ID NO: 14) |
| GN201 | MRTSQRGLSLIKSFEGLRLQAYQDSVGVWTIGYGTTRGVKAGMKISKDQA |
| | ERMLLNDVQRFEPEVERLIKVPLNQDQWDALMSFTYNLGAANLESSTLRD |
| | LLNAGNYAAAAEQFPHWNKAGGQVLAGLTRRRAAERELFLGAAGPRRPR |
| | RPGRRAPV (SEQ ID NO: 15) |
| GN205 | GPRRPRRPGRRAPVMRTSQRGLSLIKSFEGLRLQAYQDSVGVWTIGYGTRG |
| | VKAGMKISKDQAERMLLNDVQRFEPEVERLIKVPLNQDQWDALMSFTYN |
| | LGAANLESSTLRRLLNAGNYAAAAEQFPRWNKAGGQVLAGLTRRRAAER |
| | ELFLGAA (SEQ ID NO: 16) |
| GN200 | MSFKLGKRSLSNLEGVHPDLIKVVKRAIELTECDFTVTEGLRSKERQAQL |
| | LKEKKTTTSNSRHLTGHAVDLAAWVNNTVSWDWKYYYQIADAMKKAAS |
| | ELNVSIDWGGDWKKFKDGPHFELTWSKYPIKGASRKKTRKRLKKIGKVLK |
| | WI (SEQ ID NO: 17) |
| GN204 | MKLSEKRALFTQLLAQLILWAGTQDRVSVALDQVKRTQAEADANAKSGA |
| | GIRNSLHLLGLAGDLILYKDGKYMDKSEDYKFLGDYWKSLHPLCRWGGD |
| | FKSRPDGNHFSLEHEGVQRKKTRKRLKKIGKVLKWI (SEQ ID NO: 18) |
| GN150 | MSFKLGKRSLSNLEGVHPDLIKVVKRAIELTECDFTVTEGLRSKERQAQLL |
| | KEKKTTTSNSRHLTGHAVDLAAWVNNTVSWDWKYYYQIADAMKKAASE |
| | LNVSIDWGGDWKKFKDGPHFELTWSKYPIKGAS (SEQ ID NO: 19) |
| GN203 | MKLSEKRALFTQLLAQLILWAGTQDRVSVALDQVKRTQAEADANAKSGA |
| | GIRNSLHLLGLAGDLILYKDGKYMDKSEDYKFLGDYWKSLHPLCRWGGD |
| | FKSRPDGNHFSLEHEGVQ (SEQ ID NO: 20) |
| GN9 | MKNFNEIIEHVLKHEGGYVNDPKDLGGETKYGITKRFYPDLDIKNLTIEQA |
| | TEIYKKDYWDKNKVESLPQNLWHIYFDMCVNMGKRTAVKVLQRAAVNR |
| | GRDIEVDGGLGPATIGALKGVELDRVRAFRVKYYVDLITAR |
| | PEQEKFYLGWFRRATEV (SEQ ID NO: 21) |
| GN10 | MSKQGGVKVAQAVAALSSPGLKIDGIVGKATRAAVSSMPSSQKAATDKIL |
| | QSAGIGSLDSLLAEPAAATSDTFREVVLAVAREARKRGLNPAFYVAHIAL |
| | ETGWGRSVPKLPDGRSSYNYAGLKYAAVKTQVKGKTETNTLEYIKSLPKT |
| | VRDSFAVFASAGDFSRVYFWYLLDSPSAYRYPGLKNAKTAQEFGDILQKG |
| | GYATDPAYAAKVASIASTAVARYGSDVSSVA (SEQ ID NO: 22) |
| GN13 | MSDKRVEITGNVSGFFESGGRGVKTVSTGKGDNGGVSYGKHQLASNNGS |
| | MALFLESPFGAPYRAQFAGLKPGTAAFTSVYNKIANETPTAFERDQFQYIA |
| | ASHYDPQAAKLKAEGINVDDRHVAVRECVFSVAVQYGRNTSIIIKALGSNF |
| | RGSDKDFIEKVQDYRGATVNTYFKSSSQQTRDSVKNRSQQEKQMLLKLLN |
| | S (SEQ ID NO: 23) |
| GN17 | MTLRYGDRSQEVRQLQRRLNTWAGANLYEDGHFGAATEDAVRAFQRSH |
| | GLVADGIAGPKTLAALGGADCSHLLQNADLVAAATRLGLPLATIYAVNQV |
| | ESNGQGFLGNGKPAILFERHIMYRRLAAHDQVTADQLAAQFPALVNPRPG |
| | GYAGGTAEHQRLANARQIDDTAALESASWGAFQIMGFHWQRLGYISVQA |
| | FAEAMGRSESAQFEAFVRFIDTDPALHKALKARKWADFARLYNGPDYKRN |
| | LYDNKLARAYEQHANCAEASA (SEQ ID NO: 24) |
| GN105 | MAVVSEKTAGGRNVLAFLDMLAWSEGTSTIRGSDNGYNVVVGGGLFNGY |
| | ADHPRLKVYLPRYKVYSTAAGRYQLLSRYWDAYRESLALKGGFTPSNQD |
| | LVALQQIKERRSLADIQAGRLADAVQKCSNIWASLPGAGYGQREHSLDDL |
| | TAHYLAAGGVLS (SEQ ID NO: 25) |
| GN108 | MILTKDGFSIIRNELFEGKLDQTQVDAINFIVEKATEYGLTYPEAAYLLATIY |
| | HETGLPSGYRTMQPIKEAGSDSYLRSKKYYPYIGYGYVQLTWEENYERIGK |
| | LIGIDLVKNPEKALEPLIAIQIAIKGMLNGWFTGVGFRRKRPVSKYNKQQYV |
| | AARNIINGKDKAELIAKYAIIFERALRSL (SEQ ID NO: 26) |
| GN123 | MTLLKKGDKGDAVKQLQQKLKDLGYTLGVDGNFGNGTDTVVRSFQTKM |
| | KLSVDGVVGNGTMSTIDSTLAGIKAWKTSVPFPATNKSRAMAMPTLTEIGR |
| | LTNVDPKLLATFCSIESAFDYTAKPYKPDGTVYSSAEGWFQFLDATWDDE |
| | VRKHGKQYSFPVDPGRSLRKDPRANGLMGAEFLKGNAAILRPVLGHEPSD |
| | TDLYLAHFMGAGGAKQFLMADQNKLAAELFPGPAKANPNIFYKSGNIART |
| | LAEVYAVLDAKVAKHRA (SEQ ID NO: 27) |

For GN3 and GN4 (each a member of the lysozyme-like superfamily), the modified derivatives GN147 and GN146, respectively, were generated based on the introduction of two amino acid substitutions at positions equivalent to that shown (31-33) in human lysozyme to improve both in vitro (in buffer and/or media) and in vivo antibacterial activity (in an animal infection model).

The GN3 lysin polypeptide was modified to include amino acid substitutions, in particular, R101D and R117H amino acid substitutions. This resulted in the modified polypeptide, GN147. These amino acid substitutions resulted in a reduction in pI from 9.98 in the GN3 polypeptide to 9.39 in the GN147 polypeptide.

The GN4 lysin was modified to include amino acid substitutions, in particular, K99D, R115H. This resulted in the modified lysin polypeptide, GN146. These amino acid substitutions resulted in reduction in pI from 9.58 in the GN4 polypeptide to 8.01 in the GN146 polypeptide.

The positions for each mutation in GN3 and GN4 were gauged based on a rough comparison with mutations in human lysozyme (HuLYZ), as HuLYZ bears no significant homology to either GN3 or GN4 at the amino acid sequence level.

While lysins GN3 and GN4 are not similar to T4 lysozyme at the amino acid level, they are of a similar size. A line up of their structures revealed charged residues. Equivalence was therefore judged solely by the presence of a charged residue in GN3 and GN4 at roughly the same location in the primary sequence of T4 lysozyme. Again, as described above, in general, charged amino acids were substituted by ones having no charge and the mutants screened for activity.

Additional modifications of both GN3 and GN4 polypeptides were also introduced, including the addition of an N-terminal peptide sequence (GPRRPRRPGRRAPV—SEQ ID NO: 28), derived from a much larger antimicrobial peptide (AMP) described by Daniels and Schepartz, 2007 (34), to generate GN205 and GN156, respectively.

The GN lysin polypeptides may be further modified by the addition of pI modifying mutations. In an embodiment, the amino acid substitutions (R101D) and (R117H) were introduced into the GN3 lysin to generate the GN147 lysin. In another embodiment, the amino acid substitutions (K99D) and (R115H) were introduced into the GN4 lysin to generate the GN146 lysin.

The GN4 polypeptide was also modified by the addition of two different previously described N-terminal cationic AMPs, either KFFKFFKFFK (SEQ ID NO: 29) or KRKKRKKRK (SEQ ID NO: 30) (35, 36) connected to GN4 via a linker domain AGAGAGAGAGAGAGAGAS (SEQ ID NO: 31) previously described by Briers et al. 2014 (36), to generate the modified lysins GN92 and GN54, respectively.

Modifications of the lysins GN37, GN150 and GN203 (each a member of the VanY superfamily) were generated by the addition of a C-terminal AMP, RKKTRKRLKKIGKVLKWI (SEQ ID NO: 32) previously developed as a derivative of the porcine myeloid antimicrobial peptide-36 (PMAP-36) (22). The modification of GN37, GN150 and GN203 by addition of the C-terminal RI18 peptide sequence resulted in the modified derivatives GN121, GN200, and GN204, respectively. An additional modification was also included whereby the AMP (KFFKFFKFFK—SEQ ID NO: 29) (35) and linker domain (AGAGAGAGAGAGAGAGAS—SEQ ID NO: 31) (36) described above were appended to the N-terminus of GN37 to generate modified lysin GN94.

The peptides used to make GN121, GN156, GN200, GN201, GN202, GN204 and GN205 are not believed to have been used previously to modify lysins. The rationale for using them was as follows: 1) when added to the indicated lysin, the predicted secondary structure of both the AMP and lysin does not appreciably change or does not change at all (as determined using a known protein structure predicting program); 2) these peptides have been previously described in the literature as having potent activity; and 3) these AMPs were tested in serum and found potent activity. The same applies for the peptide used in GN92 and GN9. However, a linker sequence was also used in these constructs, to join the AMP and the lysin, to obtain an appropriate secondary structure of the AMP (closely resembling that of free AMP) when the AMP is fused to the lysin.

For GN54, both the AMP and the linker have been previously used to modify lysins but no reports of activity in serum have been seen in the literature. GN54, does have activity in serum.

Lysins GN3, GN9, GN10, GN13, GN17, GN105, GN108, GN123 and GN150 have been synthesized and/or produced recombinantly, and purified to (>90%) homogeneity and examined in a series of activity assays. The MIC assay was performed using Pseudononas aeruginosa cultured in two media types, CAA and CAA supplemented with 25% human serum (“CAA/HuS”). The activity of many GN lysins (including the control T4 lysozyme in Table 4) is repressed in both CAA and CAA/HuS.

For the set of 9 novel GN lysins examined here (i.e., GN3, GN9, GN10, GN13, GN17, GN105, GN108, GN123, and GN150), we observed an MIC range of 2->128 in both CAA and CAA/HuS.

Modified lysins GN54, GN92, GN94, GN121, GN146 and GN147 were each purified to (>90%) homogeneity and examined in a series of in vitro activity assays. The MIC value (in g/mL) for each of the GN lysins in CAA/HuS is as follows: GN54, 2; GN92, 4; GN94, 2; GN121, 0.5; GN146, 2; GN147, 4, as shown in Table 4.

TABLE 4
Minimal Inhibitory Concentration (MIC) analysis of Purified GN Lysins.

| | | MIC (μg/mL) | MIC (μg/mL |
|---|---|---|---|
| Lysin | Lysin Type | in CAA | in CAA/HuS |
| GN3 | Native | 16 | 16 |
| GN147 | Modified GN3 | 2 | 4 |
| GN4 | Native | 64 | 16 |
| GN146 | Modified GN4 | 2 | 2 |
| GN156 | Modified GN4 | 32 | 2 |
| GN54 | Modified GN4 | 64 | 2 |
| GN92 | Modified GN4 | 32 | 4 |
| GN37 | Native | >128 | 32 |
| GN121 | Modified GN37 | 0.5 | 0.5 |
| GN94 | Modified GN37 | 16 | 2 |
| GN9 | Native | 8 | 2 |
| GN10 | Native | 8 | 16 |
| GN13 | Native | 8 | >128 |
| GN17 | Native | 32 | 16 |
| GN105 | Native | >128 | 32 |
| GN108 | Native | 8 | 8 |
| GN123 | Native | 2 | 128 |
| GN150 | Native | 2 | 32 |
| GN126 | Native (control) | 2 | 128 |
| T4 LYZ | Native (control) | >128 | >128 |

Significantly, the MIC values (in g/mL) determined using CAA/HuS for each of the parental lysin molecules GN3, GN4, and GN37 are 16, 16 and 32, respectively; therefore, the modification of each agent resulted in an improvement of activity in human serum. T4 lysozyme (MIC=>128 μg/mL) was included as a control standard for GN lysins that are inactive in human serum. GN126 (MIC=128 μg/mL) was also included as a control, and corresponds to Art-175 (37); Art-175 is an artilysin, described in the literature, consisting of a fusion of the AMP SMAP-29 to GN lysin KZ144.

In addition to the MIC analysis, the modified GN lysins (GN54, GN92, GN94, GN121, GN146 and GN147) were also shown to have potent anti-biofilm activity, wherein the Minimal Biofilm Eradicating Concentration (MBEC) values range from 0.25-2 μg/mL, see Table 5.

Each of the GN3, GN9, GN10, GN13, GN17, GN105, GN108 and GN123 were shown to have potent antibiofilm activity with MBEC values ranging from 0.125-4 μg/mL (Table 5) and have no hemolytic activity whatsoever (Table 6). It is anticipated that the remaining modified lysins will exhibit improved activity against biofilm compared to the parent lysins and will also have reduced or eliminated hemolytic properties as well as increased activity in the presence of blood matrices including human serum.

TABLE 5
Minimal Biofilm Eradicating Concentration (MBEC)
Analysis of Purified GN Lysins

| Lysin | Lysin Type | MBEC (μg/mL) |
|---|---|---|
| GN3 | Native | 0.25 |
| GN147 | Modified GN3 | 0.25 |
| GN4 | Native | 1 |
| GN146 | Modified GN4 | 2 |
| GN156 | Modified GN4 | 0.5 |
| GN54 | Modified GN4 | n.d. |
| GN92 | Modified GN4 | 0.5 |
| GN37 | Native | 0.25 |
| GN121 | Modified GN37 | 0.25 |
| GN94 | Modified GN37 | 2 |
| GN9 | Native | 0.125 |
| GN10 | Native | 0.5 |
| GN13 | Native | 0.125 |
| GN17 | Native | 0.125 |
| GN105 | Native | 4 |
| GN108 | Native | 0.125 |
| GN123 | Native | 4 |
| GN150 | Native | 0.25 |

The modified GN lysins (GN54, GN92, GN94, GN121, GN146 and GN147) were also shown to have no hemolytic activity (MHC values of >128 ag/mL), see Table 6.

TABLE 6
Minimal Hemolytic Concentration (MHC) Analysis
of Purified GN Lysins

| Lysin | Lysin Type | MHC (μg/mL) |
|---|---|---|
| GN3 | Native | >128 |
| GN147 | Modified GN3 | >128 |
| GN4 | Native | >128 |
| GN146 | Modified GN4 | >128 |
| GN156 | Modified GN4 | >128 |
| GN54 | Modified GN4 | >128 |
| GN92 | Modified GN4 | >128 |
| GN37 | Native | >128 |
| GN121 | Modified GN37 | >128 |
| GN94 | Modified GN37 | >128 |
| GN9 | Native | >128 |
| GN10 | Native | >128 |
| GN13 | Native | >128 |
| GN17 | Native | >128 |
| GN105 | Native | >128 |
| GN108 | Native | >128 |
| GN123 | Native | >128 |
| GN150 | Native | >128 |

The modified GN lysins (GN54, GN92, GN94, GN121, GN146 and GN147) were also shown to have bactericidal activity in the time-kill format, as defined by CFU decreases of ≥3-Log₁₀ by 3 hours after the addition of lysin. See Table 7 and Table 8.

In the time-kill assay format, GN3, GN17, GN108, GN123, and GN150 each demonstrated bactericidal activity at a 3-hour timepoint after addition at a concentration of 10 μg/mL in either CAA/HuS or HEPES buffer (Tables 7 and 8, respectively).

TABLE 7
Time-Kill Analysis of Purified GN Lysin Activity in CAA/HuS

| | | Log₁₀ CFU/mL | | |
|---|---|---|---|---|
| Lysin | Lysin Type | T = 0 | T = l hr | T = 3 hr* |
| no | Buffer control | 7.8 | 7.7 | 7.2 |
| GN3 | Native | 7.8 | 5.8 | <3.7‡ |
| GN147 | Modified GN3 | 7.8 | 6.5 | 4.2‡ |
| GN4 | Native | 7.8 | 6.0 | <3.7‡ |
| GN146 | Modified GN4 | 7.8 | 5.9 | 4.0‡ |
| GN156 | Modified GN4 | 7.8 | 5.7 | <3.7‡ |
| GN54 | Modified GN4 | 7.8 | n.d. | n.d. |
| GN92 | Modified GN4 | 7.8 | 6.2 | <3.7‡ |
| GN37 | Native | 7.8 | 6.2 | <3.7‡ |
| GN121 | Modified GN37 | 7.8 | 7.4 | <3.7‡ |
| GN94 | Modified GN37 | 7.8 | 6.4 | <3.7‡ |
| GN9 | Native | 7.8 | 6.8 | 7.3 |
| GN10 | Native | 7.8 | 7.4 | 7.4 |
| GN13 | Native | 7.8 | n.d. | n.d. |
| GN17 | Native | 7.8 | 6.4 | 4.2‡ |
| GN105 | Native | 7.8 | 7.0 | 6.3 |
| GN108 | Native | 7.8 | 5.7 | <3.7‡ |
| GN123 | Native | 7.8 | 6.7 | <3.7‡ |
| GN150 | Native | 7.8 | 6.0 | <3.7‡ |

*The limit of detection is 3.7 Log10 CFU/mL.
‡indicates bactericidal activity.

TABLE 8
Time-Kill Analysis of Purified GN Lysin Activity in HEPES Buffer

| | | Log₁₀ CFU/mL | | |
|---|---|---|---|---|
| Lysin | Lysin Type | T = 0 | T = l hr | T = 3 hr* |
| no | Buffer control | 7.8 | 7.7 | 7.2 |
| GN3 | Native | 7.8 | <3.7‡ | <3.7‡ |
| GN147 | Modified GN3 | 7.8 | <3.7‡ | <3.7‡ |
| GN4 | Native | 7.8 | 5.7 | <3.7‡ |
| GN146 | Modified GN4 | 7.8 | 6.7 | <3.7‡ |
| GN156 | Modified GN4 | 7.8 | 5.7 | <3.7‡ |
| GN54 | Modified GN4 | 7.8 | n.d. | n.d. |
| GN92 | Modified GN4 | 7.8 | 5.7 | <3.7‡ |
| GN37 | Native | 7.8 | 6.3 | <3.7‡ |
| GN121 | Modified GN37 | 7.8 | <3.7‡ | <3.7‡ |
| GN94 | Modified GN37 | 7.8 | 6.0 | <3.7‡ |
| GN9 | Native | 7.8 | 6.7 | 5.7 |
| GN10 | Native | 7.8 | 5.7 | <3.7‡ |
| GN13 | Native | 7.8 | n.d. | n.d. |
| GN17 | Native | 7.8 | 5.4 | <3.7‡ |
| GN105 | Native | 7.8 | 6.6 | <3.7 |
| GN108 | Native | 7.8 | 6.4 | <3.7‡ |
| GN123 | Native | 7.8 | 5.6 | <3.7‡ |
| GN150 | Native | 7.8 | 5.7 | <3.7‡ |

*The limit of detection is 3.7 Log10 CFU/mL.
‡indicates bactericidal activity.

A subset of the GN lysins (GN4, GN37, GN108, and GN150) were examined in the checkerboard assay using CAA/HuS, and shown to synergize with a range of antibiotics and exhibit activity against Gram-negative bacteria, such as the carbapenem-resistant clinical strain, WC-452 (including amikacin, azithromycin, aztreonam, ciprofloxacin, colistin, rifampicin, tobramycin, fosfomycin, gentamicin, piperacillin or a carbapenem, e.g., imipenem, meropenem, and piperacillin (Table 9)).

The modified lysins GN92, GN121, and GN147 were also each shown to synergize with a range of antibiotics and exhibit activity against Gram-negative bacteria, such as the carbapenem-resistant clinical strain, WC-452 (including amikacin, azithromycin, aztreonam, ciprofloxacin, colistin, rifampicin, tobramycin, fosfomycin, gentamicin, piperacillin or a carbapenem, e.g., imipenem, meropenem, and piperacillin) in CAA/HuS, as shown in Table 9. These data indicate that the synergy will persist in vivo in the presence of human serum. Fractional inhibitor concentration index (FICI) values were determined for all combinations; values of <0.5 indicate synergy.

Moreover, these synergy data also indicate that, in some embodiments, the present lysins will be able to drive the resensitization of gram-negative bacteria including MDR organisms, such as carbapenem-resistant P. aeruginosa as described in the Examples. Generally resensitization occurs in synergistic combinations in which the antibiotic MIC values fall below established breakpoints, e.g., a MIC value of ≤2 for antibiotic sensitive bacteria, a MIC value of 4 for intermediately sensitive bacteria and a MIC value of ≥8 for antibiotic resistant bacteria, e.g. carbapenem-resistant isolates. See Clinical and Laboratory Standards Institute (CLSI), CLSI. 2019. M100 Performance Standards for Antimicrobial Susceptibility Testing; 29th Edition. Clinical and Laboratory Standards Institute, Wayne, Pa.

TABLE 9
Checkerboard Analysis of Purified GN Lysins with Antibiotics

| | Amikacin | Azithromycin | Aztreonam | Ciprofloxacin | Colistin | Rifampicin | Tobramycin |
|---|---|---|---|---|---|---|---|
| GN4 | 0.531 | 0.094 | 0.156 | 0.250 | 0.156 | 0.156 | 0.375 |
| GN92 | 0.375 | 0.063 | 0.188 | 0.281 | 0.094 | 0.094 | 0.500 |
| GN147 | 0.375 | 0.250 | 0.188 | 0.281 | 0.188 | 0.281 | 0.5 |
| GN37 | 0.125 | 0.188 | 0.531 | 0.281 | 0.156 | 0.281 | 0.156 |
| GN121 | 0.375 | 0.188 | 0.625 | 0.313 | 0.375 | 0.313 | 0.188 |
| GN108 | 0.156 | 0.060 | 0.250 | 0.281 | 0.133 | 0.125 | 0.188 |
| GN150 | 0.313 | 0.125 | 0.188 | 0.250 | 0.094 | 0.094 | 0.500 |
| Lysin | Fosfomycin | Gentamicin | Imipenem | Meropenem | Piperacillin | | |
| GN37 | 0.313 | 0.313 | 0.313 | 0.500 | 0.375 | | |
| GN108 | 0.188 | 0.188 | 0.039 | 0.250 | 0.531 | | |
| GN4 | 0.250 | 0.375 | 0.125 | n.d. | 0.313 | | |
| GN92 | 0.500 | 0.375 | 0.125 | 0.375 | 0.500 | | |
| GN147 | 0.250 | 0.375 | 0.375 | 0.375 | 0.188 | | |
| GN121 | 0.375 | 0.375 | 0.375 | 0.313 | 0.375 | | |
| GN123 | 0.250 | 0.375 | 0.188 | 0.125 | 0.500 | | |
| GN150 | 0.375 | 0.375 | 0.375 | n.d. | 0.500 | | |

Based on the activity of specific GN lysins in the presence of human serum (and on the nature of their amino acid sequence and homology to other lysins as well as protein expression and purification profiles), it is anticipated that lysins GN3, GN9, GN10, GN13, GN17, GN105, GN108, GN123, GN150 and 203 are superior candidates for further development in either their native lysin form or after further modification in the manner described herein, i.e., with substitution of typically 1 to 3 charged amino acid residues with non-charged residues (and maintenance of activity in the absence and presence of human serum) and/or fusion at the N- or C-terminal to an AMP peptide having an alpha helical structure.

The modified lysins corresponding to GN200-GN205 are still under analysis and may have activities similar to GN54, GN92, GN94, GN121, GN146 and GN147.

GN-lysins have been identified with varying levels of activity in the presence of human serum. Additionally, modified GN-lysins were obtained and are demonstrated to exhibit improved activity in the presence of human serum compared to that of the parental lysins or a known lysozyme (T4) or a known artilysin (GN126). Moreover, the present lysins, in synergy with antibiotics as described herein, may be used to resensitize antibiotic-resistant bacteria.

Specific embodiments disclosed herein may be further limited in the claims using “consisting of” and/or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein. The applicants reserve the right to disclaim any embodiment or feature described herein.

### EXAMPLES

### Example 1. Bacterial Strains and Growth Conditions

Antibacterial screening was performed using a P. aeruginosa clinical isolate (CFS-1292) from human blood obtained from the Hospital for Special Surgery in New York (provided by Dr. Lars Westblade, Professor of Pathology and Laboratory Medicine). Strain CFS-1292 was cultured in either lysogeny broth (LB; Sigma-Aldrich), casamino acid (CAA) media (5 g/L casamino acids, Ameresco/VWR; 5.2 mM K₂HPO₄, Sigma-Aldrich; 1 mM MgSO₄, Sigma-Aldrich) or CAA supplemented with 25% human serum (Type AB, male, pooled; Sigma-Aldrich). For purposes of the present disclosure the particular isolate of P. aeruginosa is not important and a commercially available isolate could have been used in the present experiments.

### Example 2. Gene Synthesis and Cloning

All lysins and modified lysins were synthesized as gBlocks (IDT Technologies) and cloned into the arabinose-inducible expression vector pBAD24 (24) by overlap extension PCR or through the ligation of compatible cohesive ends. All constructs were transformed into the E. coli strain TOP10 (Thermo Fisher Scientific). Other commercially available expression vectors and systems could have been employed.

### Example 3. Identification of Lysins with Intrinsic Activity

A set of up to 250 putative lysins and lysin-like enzymes were identified in the GenBank database of P. aeruginosa genomic sequences. Three search methods were used: i) a targeted BLASTp screen of all P. aeruginosa genomes using query sequences of known lysins, ii) a keyword-based search of all annotated P. aeruginosa genomes, focused on all Superfamily designations associated with lysin (and cell wall hydrolase) catalytic and binding domains; and iii) a visual search among phage sequences of non-annotated genomes for lysin-like genes. Once identified, the lysin sequences were synthesized as gBlocks, cloned into pBAD24 and transformed into E. coli TOP10 cells. The E. coli clones were then examined in a primary antibacterial activity screen (against live P. aeruginosa) using an agar overlay plate-based method (11, 13) with a modification to allow detection of GN lysin activity in overlays comprised of soft agar suspended in 50 mM Tris buffer pH7.5. A set of 109 lytic clones were identified and selected for expression and purification.

### Example 4. Expression and Purification of Lysins and Modified Lysins

A wide variety of host/expression vector combinations may be employed in expressing the polynucleotide sequences encoding lysin polypeptides of the present disclosure. Large numbers of suitable vectors are known to those of skill in the art, and are commercially available. Examples of suitable vectors are provided in Sambrook et al, eds., Molecular Cloning: A Laboratory Manual (3rd Ed.), Vols. 1-3, Cold Spring Harbor Laboratory (2001). Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. Furthermore, said vectors may provide for the constitutive or inducible expression of lysin polypeptides of the present disclosure. More specifically, suitable vectors include but are not limited to derivatives of SV40 and known bacterial plasmids, e.g., E. coli plasmids coIEl, pCRI, pBR322, pMB9 and their derivatives, plasmids such as RP4, pBAD24 and pBAD-TOPO; phage DNAS, e.g., the numerous derivatives of phage k, e.g., NM989, and other phage DNA, e.g., M13 and filamentous single stranded phage DNA; yeast plasmids such as the 2 D plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. Many of the vectors mentioned above are commercially available from vendors such as New England Biolabs, Addgene, Clontech, Life Technologies etc. many of which also provide suitable host cells).

Additionally, vectors may comprise various regulatory elements (including promoter, ribosome binding site, terminator, enhancer, various cis-elements for controlling the expression level) wherein the vector is constructed in accordance with the host cell. Any of a wide variety of expression control sequences (sequences that control the expression of a polynucleotide sequence operatively linked to it) may be used in these vectors to express the polynucleotide sequences encoding lysin polypeptides. Useful control sequences include, but are not limited to: the early or late promoters of SV40, CMV, vaccinia, polyoma or adenovirus, the lac system, the trp system, the TAC system, the TRC system, the LTR system, the major operator and promoter regions of phage k, the control regions of fd coat protein, the promoter for 3-phosphoglycerate kinase or other glycolytic enzymes, the promoters of acid phosphatase (e.g., Pho5), the promoters of the yeast-mating factors, E. coli promoter for expression in bacteria, and other promoter sequences known to control the expression of genes of prokaryotic or eukaryotic cells or their viruses, and various combinations thereof.

A wide variety of host cells are useful in expressing the lysin polypeptides of the present disclosure. Nonlimiting examples of host cells suitable for expression of lysin polypeptides of the present disclosure include well known eukaryotic and prokaryotic hosts, such as strains of E. coli, Pseudonionas, Bacillus, Streptomyces, fungi such as yeasts, and animal cells, such as CHO, Rl.l, B-W and L-M cells, African Green Monkey kidney cells (e.g., COS 1, COS 7, BSC1, BSC40, and BMT10), insect cells (e.g., Sf9), and human cells and plant cells in tissue culture. While the expression host may be any known expression host cell, in a preferred embodiment the expression host is one of the strains of E. coli. These include, but are not limited to commercially available E. coli strains such as Top 10 (Thermo Fisher Scientific), DH5oc (Thermo Fisher Scientific), XL1-Blue (Agilent Technologies), SCS110 (Stratagene), JM109 (Promega), LMG194 (ATCC), and BL21 (Thermo Fisher Scientific). There are several advantages of using E. coli as a host system including: fast growth kinetics, where under the optimal environmental conditions, its doubling time is about 20 min (Sezonov et al., J. Bacteriol. 189 8746-8749 (2007)), easily achieved high density cultures, easy and fast transformation with exogenous DNA, etc. Details regarding protein expression in E. coli, including plasmid selection as well as strain selection are discussed in details by Rosano, G. and Ceccarelli, E., Front Microbiol., 5: 172 (2014).

Efficient expression of lysin polypeptides and vectors thereof depends on a variety of factors such as optimal expression signals (both at the level of transcription and translation), correct protein folding, and cell growth characteristics. Regarding methods for constructing the vector and methods for transducing the constructed recombinant vector into the host cell, conventional methods known in the art can be utilized. While it is understood that not all vectors, expression control sequences, and hosts will function equally well to express the polynucleotide sequences encoding lysin peptides of the present disclosure, one skilled in the art will be able to select the proper vectors, expression control sequences, and hosts without undue experimentation to accomplish the desired expression without departing from the scope of this disclosure. In some embodiments, a correlation has been found between level of expression and activity of the expressed polypeptide; in E. coli expression systems in particular, moderate levels of expression (for example between about 1 and 10 mg/liter) have produced lysin polypeptides with higher levels of activity than those that were expressed at higher levels in in E. coli (for example between about 20 and about 100 mg/liter), the latter having sometimes produced wholly inactive polypeptides.

Lysin polypeptides of the present disclosure can be recovered and purified from recombinant cell cultures by well-known methods including without limitation ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography can also employed for lysin polypeptide purification.

Alternatively, the vector system used for the production of lysin polypeptides of the present disclosure may be a cell free expression system. Various cell free expression systems are commercially available, including, but are not limited to those available from Promega, LifeTechnologies, Clonetech, etc.

Protein solubilization and purification (using one or more chromatographic techniques) are performed in a well-buffered solution containing a suitable ionic strength of a monovalent salt, e.g., an ionic strength equivalent to 300-500 mM of NaCl.

Immobilized metal affinity chromatography (IMAC) is preferably used as the initial purification step. If additional purification is required, size-exclusion chromatography (gel filtration) can be used in a further step. If necessary, ion exchange chromatography can be used as a final step.

A range of induction times and temperatures were used to identify optimal conditions for protein expression and purification. The main methodologies are described in previous studies (11, 13, 25). Briefly, replicates of each expression clone were induced in both LB and RM media (Thermo Fisher Scientific) over at 2-24 hour period at 24° C.-37° C. The induced cultures were then pelleted and disrupted using BugBuster (Millipore Sigma) before an assessment of soluble protein expression was made by SDS-PAGE and Coomassie staining. The optimal condition for expression of each lysin was then used to scale up production. The purifications were performed using either anion exchange (HiTrap DEAE FF), cation exchange (HiTrap Capto MMC), hydrophobic interaction columns (HiTrap Phenyl FF), and/or size exclusion columns (HiLoad 16/600 SuperDex) with the Akta™ Pure FPLC system running Unicorn 6.3 software. The addition of Mg²⁺ was sometimes used to improve solubility and increase binding capacity to the chromatographic resins. During purification, the target GN lysin was identified by molecular weight using a reducing SDS Page gel. After the last purification step, fractions containing the GN lysin of interest were pooled, buffer exchanged to 25 mM Tris 150 mM sodium chloride with pH value ranging from 7.2 to 9.0 (depending on the pI of the protein) and concentrated to about 2 mg/mL. Concentration was measured by NanoDrop and protein was stored at −80° C. in 500 μL aliquots.

### Example 5. Determination of Minimal Inhibitory Concentration (MIC)

The minimal inhibitory concentration of each GN lysin against P. aeruginosa was determined using a modification of the standard broth microdilution reference method defined by the Clinical and Laboratory Standards Institute (CLSI)(26). The modification was based on the replacement of Mueller Hinton Broth with either CAA media or CAA supplemented with 25% human serum.

### Example 6. Determination of Minimal Biofilm Eradicating Concentration (MBEC)

The MBEC of CF-301 was determined using a variation of the broth microdilution MIC method with modifications (27, 28). Here, fresh colonies of P. aeruginosa strain ATCC 17647 were suspended in PBS (0.5 McFarland units), diluted 1:100 in TSBg (tryptic soy broth supplemented with 0.2% glucose), added as 0.15 ml aliquots to a Calgary Biofilm Device (96-well plate with a lid bearing 96 polycarbonate pegs; Innovotech) and incubated 24 hours at 37° C. Biofilms were washed and treated with a 2-fold dilution series of CF-301 in TSBg at 37° C. for 24 hours. All samples were examined in triplicate. After treatment, wells were washed, air-dried at 37° C., and stained with 0.05% crystal violet for 10 minutes. After staining, the biofilms were destained in 33% acetic acid and the OD₆₀₀ of extracted crystal violet was determined. The MBEC of each sample was the minimum drug concentration required to remove >95% of the biofilm biomass assessed by crystal violet quantitation.

### Example 7. Checkerboard Assay to Examine Synergy with Antibiotics

The checkerboard assays is based on a modification of the CLSI method for MIC determination by broth microdilution (26, 29). Checkerboards were constructed by first preparing columns of a 96-well polypropylene microtiter plate, in which each well had the same amount of antibiotic diluted 2-fold along the horizontal axis. In a separate plate, comparable rows were prepared in which each well had the same amount of GN lysin diluted 2-fold along the vertical axis. The GN lysin and antibiotic dilutions were then combined, so that each column had a constant amount of antibiotic and doubling dilutions of GN lysin, while each row had a constant amount of GN lysin and doubling dilutions of antibiotic. Each well thus had a unique combination of GN lysin and antibiotic. Bacteria were added to the drug combinations at concentrations of 1×10⁵ CFU/mL in CAA with 25% human serum. The MIC of each drug, alone and in combination, was then recorded after 16 hours at 37° C. in ambient air. Summation fractional inhibitory concentrations (ΣFICs) were calculated for each drug and the minimum ΣFIC value (ΣFICmin) was used to determine synergy. ΣFICs were calculated as follows: ΣFIC=FIC A+FIC B, where FIC A is the MIC of each antibiotic in the combination/MIC of each antibiotic alone, and FIC B is the MIC of each GN lysin in the combination/MIC of each GN lysin alone. The combination is considered synergistic when the ΣFIC is ≤0.5, strongly additive when the ΣFIC is >0.5 to <1, additive with the ΣFIC is 1−<2, and antagonistic when the ΣFIC is ≥2.

### Example 8. Assay of GN Lysin Hemolytic Activity

The hemolytic activity of the GN lysins was measured as the amount of hemoglobin released by the lysis of human erythrocytes (30). Briefly, 3 ml of fresh human blood cells (hRBCs) obtained from pooled healthy donors (BioreclamationIVT) in a polycarbonate tube containing heparin was centrifuged at 1,000×g for 5 min at 4° C. The erythrocytes obtained were washed three times with phosphate-buffered saline (PBS) solution (pH 7.2) and resuspended in 30 PBS. A 50 μl volume of the erythrocyte solution was incubated with 50 μl of each GN lysin (in PBS) in a 2-fold dilution range (from 128 μg/mL to 0.25 μg/mL) for 1 h at 37° C. Intact erythrocytes were pelleted by centrifugation at 1,000×g for 5 min at 4° C., and the supernatant was transferred to a new 96-well plate. The release of hemoglobin was monitored by measuring the absorbance at 570 nm. As a negative control, hRBCs in PBS were treated as above with 0.1% Triton X-100.

### Example 9. Time-Kill Assay of GN Lysin Activity

An overnight culture of P. aeruginosa was diluted 1:50 into fresh CAA media and grown for 2.5 hours at 37° C. with agitation. Exponential phase bacteria were then pelleted and resuspended in 1/5 culture volume of 25 mM HEPES, pH7.4 before a final adjustment to an optical density corresponding to a McFarland value of 0.5. The adjusted culture was then diluted 1:50 into either 25 mM HEPES pH7.4 or CAA/HuS and the GN lysins were added at a final concentration of 10 μg/mL. Control cultures were included with the addition of no lysin (i.e., buffer control). All treatments were incubated at 37° C. with aeration. At time points before the addition of lysin (or buffer control) and at 1 hour and 3 hours intervals thereafter, culture samples were removed for quantitative plating on CAA agar plates.

### Example 10. Resensitization of Carbapenem-Resistant Clinical Strains Using Antibiotics in Combination with Lysins

The ability of GN121 or GN123 to resensitize carbapenem-resistant P. aeruginosa strains to carbapenems was assessed by combining each of the foregoing lysins with two carbapenems, i.e., imipenem (IPM) or meropenem (MEM). Up to seven carbapenem-resistant isolates were assessed. Resensitization occurs in synergistic combinations in which the carbapenem MIC values fall below established breakpoints, e.g. a MIC value of ≤2 for carbapenem-sensitive isolates, a MIC value of 4 for intermediately sensitive carbapenem isolates and a MIC value of ≥8 for carbapenem-resistant isolates. See Clinical and Laboratory Standards Institute (CLSI), CLSI. 2019. M100 Performance Standards for Antimicrobial Susceptibility Testing; 29th Edition. Clinical and Laboratory Standards Institute, Wayne, Pa.

As indicated in Tables 10-13 synergistic combinations with GN123 or GN121 demonstrated reductions of IPM and MEM MICS to below breakpoint values for each of the seven carbapenems examined. These observations are consistent with resensitization. This novel ability of lysins to resensitize antibiotic-resistant strains to conventional antibiotics indicates the benefit of these biologics as therapeutics to combat and reverse antimicrobial resistance.

TABLE 10
Gram-negative bacterial resensitization using a combination
of IMIPENEM and GN123

| | IMIPENEM MIC (μg/mL) | | GN123 (μg/mL) | | |
|---|---|---|---|---|---|
| Isolate | Alone | Combination | Alone | Combination | FICI |
| PA19 | 32 (R) | 0.5 (S) | 8 | 0.125 | 0.03 |
| Analysis of additional CARBAPENEM^{R} isoates: | | | | | |
| PA20 | 16 (R) | 1 (S) | 16 | 2 | 0.188 |
| PA21 | 32 (R) | 0.5 (S) | 8 | 1 | 0.141 |
| PA22 | 16 (R) | 2 (S) | 16 | 1 | 0.188 |
| PA23 | 8 (R) | 0.25 (S) | 8 | 2 | 0.281 |
| PA24 | 32 (R) | 2 (S) | 16 | 2 | 0.188 |
| WC-452 | 16 (R) | 1 (S) | 16 | 2 | 0.188 |

(R) = resistant (S) = sensitive

TABLE 11
Gram-negative bacterial resensitization using a combination
of MEROPENEM and GN123

| | MEROPENEM MIC (μg/mL) | | GN123 (μg/mL) | | |
|---|---|---|---|---|---|
| Isolate | Alone | Combination | Alone | Combination | FICI |
| PA19 | 32 (R) | 0.5 (S) | 8 | 0.25 | 0.046 |
| PA20 | 16 (R) | 0.5 (S) | 16 | 1 | 0.094 |
| PA21 | 32 (R) | 1 (S) | 8 | 1 | 0.156 |
| PA22 | 16 (R) | 1 (S) | 16 | 1 | 0.125 |
| PA23 | 16 (R) | 0.5 (S) | 8 | 1 | 0.156 |
| PA24 | 32 (R) | 2 (S) | 16 | 0.5 | 0.094 |
| WC-452 | 16 (R) | 1 (S) | 16 | 1 | 0.125 |

(R) = resistant (S) = sensitive

TABLE 12
Gram-negative bacterial resensitization using a combination
of IMIPENEM and GN121

| | Imipenem MIC (μg/mL) | | GN121 (μg/mL) | | |
|---|---|---|---|---|---|
| Isolate | Alone | Combination | Alone | Combination | FICI |
| PA19 | 32 (R) | 1 (S) | 1 | 0.125 | 0.155 |
| PA20 | 16 (R) | 0.5 (S) | 1 | 0.25 | 0.265 |
| PA21 | 32 (R) | 1 (S) | 1 | 0.125 | 0.155 |
| PA22 | 32 (R) | 2 (S) | 2 | 0.25 | 0.188 |
| PA23 | 16 (R) | 0.125 (S) | 1 | 0.25 | 0.257 |
| PA24 | 32 (R) | 1 (S) | 1 | 0.125 | 0.155 |

(R) = resistant (S) = sensitive

TABLE 13
Gram-negative bacterial resensitization using a combination
of MEROPENEM and GN121

| | Meropenem MIC (μg/mL) | | GN121 (μg/mL) | | |
|---|---|---|---|---|---|
| Isolate | Alone | Combination | Alone | Combination | FICI |
| PA19 | 32 (R) | 1 | 2 | 0.5 | 0.281 |
| PA20 | 16 (R) | 1 | 2 | 0.5 | 0.313 |
| PA21 | 32 (R) | 2 | 1 | 0.125 | 0.188 |
| PA22 | 16 (R) | 1 | 1 | 0.25 | 0.313 |
| PA23 | 16 (R) | 2 | 2 | 0.5 | 0.375 |
| PA24 | 32 (R) | 1 | 1 | 0.125 | 0.156 |
| WC-452 | 16 (R) | 1 | 1 | 0.06 | 0.123 |

(R) = resistant;
(S) = sensitive

Planned Experiments In Vivo

One or more experiments to test in vivo activity of the present polypeptides are currently in progress as follows:

A. Pilot PK Screening and Efficacy in the Mouse Model for Acute Lethal Bacteremia

To identify GN lysins that have systemic exposure, PK screening will be performed in CD1 mice treated with a GN lysin administered as a single IV injection. Blood PK profiles will be analyzed for up to 10 GN lysins using a research grade bioanalytical assay qualified in mouse serum. It is anticipated that multiple GN lysins will be identified that have an appropriate PK profile. Candidates that demonstrate blood exposure that achieves an AUC/MIC concentration greater than 1 will be tested in a systemic infection model. For the model, P. aeruginosa strains (PAO1 and other clinical isolates) will be re-suspended in hog gastric mucin and administered by intraperitoneal administration into CD1 mice at an inoculum that produces over 24-48 hours complete morbidity and mortality in control mice. Mice will be dosed with vehicle or a GN lysin by intravenous (IV) injection in the lateral tail vein 2 hours post lethal challenge. Morbidity and mortality will be assessed over 72 hours. It is anticipated that at appropriate dose concentrations, GN lysin treated mice will display reduced morbidity and mortality compared to vehicle controls. Studies may involve co-administration of antibiotics. A. Survival data will be analyzed by Kaplan Meyer Survival analysis using GraphPad prism and an effective concentration of 50% (EC50) calculated for each molecule. It is anticipated that multiple GN lysins with in vivo activity will be identified.

B. GN Lysin Efficacy in an Established Murine Models of Invasive Infection

The goal of this experiment and other mouse models of infection, such as lung and kidney, is to generate efficacy data in these models. The efficacy models proposed in this sub aim generally utilize mouse infections in tissues (thigh, lungs or kidneys) with bacteria. Following treatment with the lysin the bacterial burden in the tissues will be quantitated (CFU/gram of tissue) at the end of the experiment to assess robustness of treatment. In addition, each of these models can be used to define the PK/PD indices and magnitude for efficacy, e.g., AUC/MIC. These models will thus be used to evaluate the efficacy of representative GN anti-pseudomonas lysins in a murine model of pulmonary infection alone or in combination with an antibiotic to determine the best GN lysin candidates for further in vivo testing and development. Similar models can be established using Acinetobacter baumannii and used to test the efficacy of the present lysins.

B.1 Murine Neutropenic Thigh Infection and Lung Infection Models Thigh Infection Model

To establish the first model, CD-1 mice (n=12) are rendered neutropenic by the administration of cyclophosphamide 20 mg/mL administered intraperitoneally following a dosing regimen that provides greater than 99% reduction in neutrophil counts (150 mg/kg on day −4 and 100 mg/kg on day −1). A thigh infection is established by intramuscular (IM) injection into both lateral thigh muscles of an appropriate inoculum of P. aeruginosa (3×10⁴ or 1×10⁵ or 3×10⁵ or 1×10⁶ cfu/thigh) 24 hours after the second dose of immunosuppressive agent. Mice are infected while under inhaled anesthesia by intramuscular injection into both lateral thigh muscles. Each thigh can receive approximately 1.4×10⁵ CFU A. baumannii NCTC 13301 (and/or an equivalent amount of a P. aeruginosa strain). But adjustments to the amount of inoculum can be made if testing indicates this is appropriate.

Groups of 4 mice (6, vehicle only (end group)) are administered test GN lysin or vehicle or control lysin by intravenous (iv) injection at 2, 6 and 10 h post-infection. At 2 h post-infection a control group of 4 animals are humanely euthanized using pentobarbitone overdose to provide a pre-treatment control group (start). 16 hours post infection all remaining groups are humanely euthanized by pentobarbitone. Both thighs from each animal are removed and weighed individually (regarded as two independent evaluations). The lysins that will be tested would include native and modified lysins that have promising properties, i.e., substantial lytic activity and maintenance of substantial activity in the presence of blood matrices. The dosage range tested in this and other experiments detailed herein will be within the broad range of 0.01 to 500 mg/kg, but the upper limit may depend on toxicity and the lower limit maybe higher depending on intrinsic activity. Examples of lysins to be tested include GN108, GN121, GN123, and GN156.

Individual thigh tissue samples are homogenized in ice cold sterile phosphate buffered saline. Thigh homogenates are then quantitatively cultured onto CLED agar and incubated at 37° C. for 24 hours before colonies are enumerated. It is anticipated that lysins will result in a substantial reduction or elimination of bacterial colonies.

Mouse Lung Infection Model

Groups of up to 8 anaesthetized (IP injection of 100 mg/kg ketamine/6 mg/kg xylazine mixture) mice per treatment are infected by intranasal instillation of 20 μl inoculum into each nostril (5 min between nostrils) and kept in an upright position for ˜10 minutes post-infection. The strength and amount of an appropriate inoculum is previously determined as described above.

The inoculum concentration is ˜2.5×10⁶ cfu/ml (1.0×10⁵ cfu/lung) for P. aeruginosa ATCC 27853 or ˜8.8×10⁸ cfu/ml (3.5×10⁷ cfu/lung) for A. baumannii NCTC 13301. Lysins (for example the lysins identified in the preceding experiment) are dosed using the same route of administration and dosing guidelines and lungs removed and prepared for counting as per the thigh model. Colonies are enumerated following incubation at 37° C. for 24 h. Efficacy will be assessed in terms of weight of mice and bacterial burden of the lung homogenates. It is anticipated that the lysins will perform satisfactorily in abating infection as measured by substantially reduced or eliminated bacterial colonies.

B.2 Neutropenic Murine Lung Infection Model

Neutropenic BALB/c mice will be inoculated with P. aeruginosa bacteria containing an inoculum sufficient to establish a lung infection via intranasal instillation under anesthesia. Groups of 4 mice (6, vehicle only (end group)) are administered GN lysin, vehicle or control lysin by subcutaneous (SC) injection at 2, 6 and 10 h post-infection. At 2 h post-infection a control group of 4 animals are humanely euthanized using pentabarbitone overdose to provide a pre-treatment control group (start). 16 hours post infection all remaining groups are humanely euthanized by pentabarbitone. The animals are weighed and both lungs from each animal are removed and weighed individually. The lysins and dosing may be the same as described above.

Individual lung tissue samples are homogenized in ice cold sterile phosphate buffered saline. Thigh homogenates are then quantitatively cultured onto CLED agar and incubated at 37° C. for 24 hours before colonies are enumerated. The efficacy of the treatment is assessed in terms of weight and bacterial burden.

Groups of up to 8 anaesthetized (IP injection of 100 mg/kg ketamine/6 mg/kg xylazine mixture) mice per treatment are infected by intranasal instillation of P. aeruginosa inoculum into each nostril (5 min between nostrils) and kept in an upright position for ˜10 minutes post-infection. The mice are previously immunosuppressed with cyclophosphamide administered subcutaneously at 200 mg/kg on day −4 and 150 mg/kg on day −1. Infection takes place 24 hrs after the second immunosuppression dose.

The starting inoculum concentration may be ˜2.5×10⁶ cfu/ml (1.0×10⁵ cfu/lung) for P. aeruginosa ATCC 27853. Adjustments to the inoculum may be made, aiming to produce an increase in untreated mouse bacterial burden of about 1 log 10 cfu/g lung. For the survival studies described below, an inoculum will be selected that will lead to death in 24 to 72 hours.

Lysins are then dosed intranasally, mice are euthanized, weighed, the lungs extracted and weighed, and lungs removed and prepared for counting as per the thigh model. Colonies are enumerated following incubation at 37° C. for 24 h. The same lysins and dosing may be used as above. Lysins will be administered intravenously at 5 ml/kg.

In a related experiment, a suboptimal dose of an antibiotic having activity against Gram-negative bacteria, will be selected and used at a subthreshold level together with lysin. An appropriate subthreshold level can be established by treating infected mice with various doses of the antibiotic which doses are below, at and above the minimum efficacious dose. Control mice will be treated with various doses of vehicle alone. There will be one vehicle as a stand-in for the lysin treatment and another vehicle as a stand-in for the antibiotic. 40 mice will be used (5 per group) for each lysin being tested.

If imipenem is the antibiotic, a suitable subthreshold dose is likely to be between 10 and 100 mg/kg (more generally, the subthreshold or suboptimal dose may be one that effects a 1 or 2 log reduction of bacterial burden) and will be administered for example at 5 ml/kg subcutaneously or intravenously for this and the combination (antibiotic+lysin) experiments.

For the combination experiment, it is contemplated that the dose of antibiotic (for example imipenem) will be the maximum subthreshold dose tested. An appropriate dose of lysin will be determined by testing different doses of lysin in the combination treatment to see where a synergistic effect occurs. An optimum set of amounts of lysin and antibiotic will them be selected. The lysin and first treatment of antibiotic will be administered 2 hours post-infection; the second antibiotic treatment will be administered 6 hours post-infection. Tissue will be harvested 9 hours post-infection.

A similar study will be conducted using the same mouse pulmonary infection model but only mouse survival will be assessed. Infected mice will be administered lysin (or vehicle or control lysin) at 24 hours post-infection. It is contemplated that three different doses of each lysin will be used. Imipenem (or vehicle) will be administered 6 hours after the lysin dosing. The experiment will end 72 hours after infection. It is contemplated that the survival experiment will use 7 mice per group, i.e., 63 mice for each lysin tested. It is anticipated that the percentage survival will be superior when the combination is administered.

C. PK/PD Analysis in a Murine Infection Model

Animal experiments with anti-infectives that delineate the PK/PD variables (e.g., Cmax/MIC, AUC/MIC or % Time/MIC) most closely linked to efficacy are highly predictive of clinical success (31). Dose fractionation is employed to determine the PK/PD parameter associated with efficacy. By fractionating a single total dose into once a day (q24h), twice a day (ql2h), or four times a day (q6h) dosing multiple values for Cmax and free drug Time>MIC (fT>MIC) can be attained while maintaining a constant AUC. Fractionation of multiple doses generates unique exposure profiles that, when compared to efficacy endpoints, enables differentiation of Cmax/MIC, fT>MIC and AUC/MIC as the PK index and magnitude required for efficacy.

GN lysins with robust activity in one or more murine infection models identified above will undergo PK/PD analysis. PK studies will be conducted to generate multiple PK profiles and modeled to cover ranges of Cmax/MIC, AUC/MIC and fT>MIC. Dose fractionation studies will be conducted in a pulmonary efficacy model as described above (mouse rat or rabbit). The tissue bacterial burden will be utilized as the PD endpoint and data will be analyzed by plotting the CFU/g tissue as a function of different PK/PD parameters. Nonlinear regression analysis will determine which PK/PD parameter is important for efficacy. These data will be used to inform doses for non-clinical activities.

One way of conducting the PK study is the following:

TABLE 14

| | | Time point of | Number of | Total |
|---|---|---|---|---|
| Dose Level | Route of | sample | animals/time | number |
| (mg/kg) | administration | collection (h) | point | of mice |
| 10 | I | 0.083, 0.25, 0.5, 1, | 3 | 24 |
| | V | 2, 4,8,24 | | |
| 30 | I | 0.083, 0.25, 0.5, 1, | 3 | 24 |
| | V | 2, 4,8,24 | | |
| 10 | I | 0.083, 0.25, 0.5, 1, | 3 | 24 |
| 0 | V | 2, 4,8,24 | | |

At the times indicated in Table 14, mice will be euthanized and groups of three mice per time point will have blood samples collected by cardiac puncture. Separated plasma samples will be divided into two aliquots. Following blood collection, 3× bronchoalveolar lavage samples (PBS) will be collected from a narrow transverse opening made in the trachea. The three BAL samples will be combined and centrifuged to remove cellular debris. BAL supernatant will be divided into two aliquots. One sample of plasma and BAL will be further tested for lysin content and bacterial burden. The second sample will be analyzed for urea content to calculate the dilution of epithelial lining fluid (ELF) during collection of BAL.

D. Monitoring for Development of Resistance In Vivo

To identify a potential for development of resistance, in vivo homogenates from the mouse efficacy studies will be subjected to MIC analysis. If a greater than 2-fold increase in MIC is observed, the bacteria will be plated, and colonies isolated for whole genome sequencing.

### Embodiments

A. A pharmaceutical composition comprising: an isolated lysin polypeptide selected from the group consisting of one or more of

GN147, GN146, GN156, GN92, GN54, GN201, GN202, GN121, GN94, GN200, GN204, GN205 or a fragment thereof having lytic activity, or a variant thereof having lytic activity and having at least 80% sequence identity with said lysin polypeptide, and a pharmaceutically acceptable carrier, wherein the lysin polypeptide or fragment or variant is in an amount effective to inhibit the growth, or reduce the population, or kill P. aeruginosa and optionally at least one other species of Gram-negative bacteria.

B. A pharmaceutical composition comprising an effective amount of an isolated lysin polypeptide selected from the group consisting of one or more of

GN3, GN13, GN17, GN9, GN10, GN105, GN108, GN123, GN150, GN203, or a fragment thereof having lytic activity, or a variant thereof having lytic activity and having at least 80% sequence identity with said lysin polypeptide, and a pharmaceutically acceptable carrier, wherein the lysin polypeptide is in an amount effective to inhibit the growth, or reduce the population, or kill P. aeruginosa and optionally at least one other species of Gram-negative bacteria; and a pharmaceutically acceptable carrier.

C. The pharmaceutical composition of embodiment A or B, which is a solution, a suspension, an emulsion, an inhalable powder, an aerosol, or a spray.

D. The pharmaceutical composition of embodiment B further comprising one or more antibiotics suitable for the treatment of Gram-negative bacteria.

E. A vector comprising an isolated polynucleotide comprising a nucleic acid molecule that encodes a lysin polypeptide of embodiment A or B, wherein the encoded lysin polypeptide inhibits the growth, or reduces the population, or kills P. aeruginosa and optionally at least one other species of Gram-negative bacteria or a complementary sequence of said polynucleotide.

F. A recombinant expression vector comprising a nucleic acid encoding a lysin polypeptide comprising an amino acid sequence of a polypeptide according to embodiment A or B wherein the encoded lysin polypeptide has the property of inhibiting the growth, or reducing the population, or killing P. aeruginosa and optionally at least one other species of Gram-negative bacteria, the nucleic acid being operatively linked to a heterologous promoter.

G. A host cell comprising the vector of embodiment E or F.

H. The recombinant vector of embodiment E or F, wherein the nucleic acid sequence is a cDNA sequence.

I. An isolated polynucleotide comprising a nucleic acid molecule that encodes a lysin polypeptide selected from the group consisting of GN147, GN146, GN156, GN92, GN54, GN201, GN202, GN121, GN94, GN200, GN204, GN205, or a fragment thereof having lytic activity, or a variant thereof having lytic activity and having at least 80% sequence identity with said lysin polypeptide, wherein the lysin polypeptide inhibits the growth, or reduces the population, or kills P. aeruginosa and optionally at least one other species of Gram-negative bacteria.

J. The polynucleotide of embodiment I which is cDNA.

K. A method of inhibiting the growth, or reducing the population, or killing of at least one species of Gram-negative bacteria, the method comprising contacting the bacteria with a pharmaceutical composition containing a lysin polypeptide selected from the group consisting of one or more of GN147, GN146, GN156, GN92, GN54, GN201, GN202, GN121, GN94, GN200, GN204, GN205, GN3, GN13, GN17, GN9, GN10, GN105, GN108, GN123, GN150, GN203 or a fragment thereof having lytic activity or a variant thereof having lytic activity and having at least 80% sequence identity with said lysin polypeptide, in an amount effective to inhibit the growth, or reduce the population, or kill P. aeruginosa and optionally at least one other species of Gram-negative bacteria.

L. A method of treating a bacterial infection caused by a Gram-negative bacteria selected from the group consisting of P. aeruginosa and optionally one or more additional species of Gram-negative bacteria, comprising administering to a subject diagnosed with, at risk for, or exhibiting symptoms of a bacterial infection, a composition containing a lysin polypeptide selected from the group consisting of one or more of GN147, GN146, GN156, GN92, GN54, GN201, GN202, GN121, GN94, GN200, GN204, GN 205, GN3, GN13, GN17, GN9, GN10, GN105, GN108, GN123, GN150, GN203 or a fragment thereof having lytic activity, or a variant thereof having lytic activity and having at least 80% sequence identity with said lysin polypeptide, in an amount effective to inhibit the growth, or reduce the population, or kill P. aeruginosa and optionally at least one other species of Gram-negative bacteria.

M. The method of embodiment L, wherein at least one species of Gram-negative bacteria is selected from the group consisting of Pseudomonas aeruginosa, Klebsiella spp., Enterobacter spp., Escherichia coli, Citrobacter freundii, Salmonella typhimurium, Yersinia pestis, and Franciscella tulerensis.

N. The method of embodiment L, wherein the Gram-negative bacterial infection is an infection caused by Pseudomonas aeruginosa.

O. A method of treating a topical or systemic pathogenic bacterial infection caused by a Gram-negative bacteria selected from the group consisting of P. aeruginosa and optionally one or more additional species of Gram-negative bacteria in a subject, comprising administering to a subject composition containing a lysin polypeptide selected from the group consisting of one or more of GN147, GN146, GN156, GN92, GN54, GN201, GN202, GN121, GN94, GN200, GN204, GN 205, GN3, GN13, GN17, GN9, GN10, GN105, GN108, GN123, GN150, GN203 or fragments thereof having lytic activity, or variants thereof having lytic activity and having at least 80% sequence identity with said lysin polypeptide, in an amount effective to inhibit the growth, or reduce the population, or kill P. aeruginosa and optionally at least one other Gram-negative bacteria.

P. A method of preventing or treating a bacterial infection comprising co-administering to a subject diagnosed with, at risk for, or exhibiting symptoms of a bacterial infection, a combination of a first effective amount of the composition containing an effective amount of selected from the group consisting of one or more of GN147, GN146, GN156, GN92, GN54, GN201, GN202, GN121, GN94, GN200, GN204, GN 205, GN3, GN13, GN17, GN9, GN10, GN105, GN108, GN123, GN150, GN203, or a fragment thereof having lytic activity, or a variant thereof having lytic activity and having at least 80% sequence identity with said lysin polypeptide, and a second effective amount of an antibiotic suitable for the treatment of Gram-negative bacterial infection.

Q. The method of embodiment P, wherein the antibiotic is selected from one or more of ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B, and colistin.

R. A method for augmenting the efficacy of an antibiotic suitable for the treatment of Gram-negative bacterial infection, comprising co-administering the antibiotic in combination with one or more lysin polypeptides selected from the group consisting of one or more of GN147, GN146, GN156, GN92, GN54, GN201, GN202, GN121, GN94, GN200, GN204, GN205, GN3, GN13, GN17, GN9, GN10, GN105, GN108, GN123, GN150, GN203 or a fragment thereof having lytic activity, or a variant thereof having lytic activity and having at least 80% sequence identity with said lysin polypeptide, wherein administration of the combination is more effective in inhibiting the growth, or reducing the population, or killing the Gram-negative bacteria than administration of either the antibiotic or the lysin polypeptide or active fragment thereof individually.

S. An isolated lysin polypeptide, selected from the group consisting of GN147, GN146, GN156, GN92, GN54, GN201, GN202, GN121, GN94, GN200, GN204, GN205, or a fragment thereof having lytic activity, or a variant thereof having lytic activity and having at least 80% sequence identity with said lysin polypeptide, wherein the lysin polypeptide inhibits the growth, or reduces the population, or kills P. aeruginosa and, optionally, at least one other species of Gram-negative bacteria.

T. A lysin polypeptide comprising a Gram-negative native lysin selected from the group consisting of GN3, GN9, GN10, GN13, GN17, GN105, GN108, GN123, GN150 AND GN203, or a fragment thereof having lytic activity, or a variant thereof having lytic activity and having at least 80% sequence identity with said lysin polypeptide, wherein the native lysin or fragment has been optionally modified by substitution of 1 to 3 charged amino acid residues with noncharged amino acid residues, the modified native lysin or fragment retaining lytic activity.

U. A lysin polypeptide comprising a Gram-negative native lysin selected from the group consisting of GN2, GN4, GN14, GN43 and GN37, or a fragment thereof having lytic activity, or a variant thereof having lytic activity and having at least 80% sequence identity with said lysin polypeptide, wherein the native lysin or variant or fragment has been modified by substitution of 1 to 3 charged amino acid residues with noncharged amino acid residues, the modified native lysin or fragment retaining lytic activity.

V. A pharmaceutical composition according to embodiment A or B wherein the lysin polypeptide is selected from the group consisting of one or more of GN156, GN121, GN108 and GN123 or active fragments thereof or variants thereof having lytic activity and having at least 80% sequence identity with said lysin polypeptide.

W. A method according to embodiment K wherein said bacteria are in a biofilm, the method effecting disruption of the biofilm.

### REFERENCES

1. Magill S S, Edwards J R, Bamberg W, Beldavs Z G, Dumyati G, Kainer M A, Lynfield R, Maloney M, McAllister-Hollod L, Nadle J, Ray S M, Thompson D L, Wilson L E, Fridkin S K, Emerging Infections Program Healthcare-Associated I, Antimicrobial Use Prevalence Survey T. 2014. Multistate point-prevalence survey of health care-associated infections. N Engl J Med 370:1198-1208.
2. Rossolini G M, Arena F, Pecile P, Pollini S. 2014. Update on the antibiotic resistance crisis. Curr Opin Pharmacol 18:56-60.
3. Potron A, Poirel L, Nordmann P. 2015. Emerging broad-spectrum resistance in Pseudomonas aeruginosa and Acinetobacter baumannii: Mechanisms and epidemiology. Int J Antimicrob Agents 45:568-585.
4. Hattemer A, Hauser A, Diaz M, Scheetz M, Shah N, Allen J P, Porhomayon J, El-Solh A A. 2013. Bacterial and clinical characteristics of health care- and community-acquired bloodstream infections due to Pseudomonas aeruginosa. Antimicrob Agents Chemother 57:3969-3975.
5. Anderson D J, Moehring R W, Sloane R, Schmader K E, Weber D J, Fowler V G, Jr., Smathers E, Sexton D J. 2014. Bloodstream infections in community hospitals in the 21st century: a multicenter cohort study. PLoS One 9:e91713.
6. Willmann M, Bezdan D, Zapata L, Susak H, Vogel W, Schroppel K, Liese J, Weidenmaier C, Autenrieth I B, Ossowski S, Peter S. 2015. Analysis of a long-term outbreak of XDR Pseudomonas aeruginosa: a molecular epidemiological study. J Antimicrob Chemother 70:1322-1330.
7. Bassetti M, Righi E. 2015. New antibiotics and antimicrobial combination therapy for the treatment of gram-negative bacterial infections. Curr Opin Crit Care 21:402-411.
8. Wittekind M, Schuch R. 2016. Cell wall hydrolases and antibiotics: exploiting synergy to create efficacious new antimicrobial treatments. Curr Opin Microbiol 33:18-24.
9. Schmelcher M, Donovan D M, Loessner M J. 2012. Bacteriophage endolysins as novel antimicrobials. Future Microbiol 7:1147-1171.
10. Schuch R, Lee H M, Schneider B C, Sauve K L, Law C, Khan B K, Rotolo J A, Horiuchi Y, Couto D E, Raz A, Fischetti V A, Huang D B, Nowinski R C, Wittekind M. 2014. Combination therapy with lysin CF-301 and antibiotic is superior to antibiotic alone for treating methicillin-resistant Staphylococcus aureus-induced murine bacteremia. J Infect Dis 209:1469-1478.
11. Schuch R, Nelson D, Fischetti V A. 2002. A bacteriolytic agent that detects and kills Bacillus anthracis. Nature 418:884-889.
12. Briers Y, Lavigne R. 2015. Breaking barriers: expansion of the use of endolysins as novel antibacterials against Gram-negative bacteria. Future Microbiol 10:377-390.
13. Lood R. 2015. Novel phage lysin capable of killing the multidrug-resistant gram-negative bacterium Acinetobacter baumannii in a mouse bacteremia model. 59:1983-1991.
14. Thandar M, Lood R, Winer B Y, Deutsch D R, Euler C W, Fischetti V A. 2016. Novel Engineered Peptides of a Phage Lysin as Effective Antimicrobials against Multidrug-Resistant Acinetobacter baumannii. Antimicrob Agents Chemother 60:2671-2679.
15. Silhavy T J, Kahne D, Walker S. 2010. The bacterial cell envelope. Cold Spring Harb Perspect Biol 2:a000414.
16. Vaara M. 1992. Agents that increase the permeability of the outer membrane. Microbiol Rev 56:395-411.
17. Gerstmans H, Rodriguez-Rubio L, Lavigne R, Briers Y. 2016. From endolysins to Artilysin(R)s: novel enzyme-based approaches to kill drug-resistant bacteria. Biochem Soc Trans 44:123-128.
18. Zhu X, Ma Z, Wang J, Chou S, Shan A. 2014. Importance of Tryptophan in Transforming an Amphipathic Peptide into a Pseudomonas aeruginosa-Targeted Antimicrobial Peptide. PLoS One 9:e114605.
19. Deslouches B, Islam K, Craigo J K, Paranjape S M, Montelaro R C, Mietzner T A. 2005. Activity of the de novo engineered antimicrobial peptide WLBU2 against Pseudomonas aeruginosa in human serum and whole blood: implications for systemic applications. Antimicrob Agents Chemother 49:3208-3216.
20. Yeaman M R, Yount N Y. 2003. Mechanisms of antimicrobial peptide action and resistance. Pharmacol Rev 55:27-55.
21. Wang J, Chou S, Xu L, Zhu X, Dong N, Shan A, Chen Z. 2015. High specific selectivity and Membrane-Active Mechanism of the synthetic centrosymmetric alpha-helical peptides with Gly-Gly pairs. Sci Rep 5:15963.
22. Lyu Y, Yang Y, Lyu X, Dong N, Shan A. 2016. Antimicrobial activity, improved cell selectivity and mode of action of short PMAP-36-derived peptides against bacteria and Candida. Sci Rep 6:27258.
23. Sanchez-Gomez S, Lamata M, Leiva J, Blondelle S E, Jerala R, Andra J, Brandenburg K, Lohner K, Moriyon I, Martinez-de-Tejada G. 2008. Comparative analysis of selected methods for the assessment of antimicrobial and membrane-permeabilizing activity: a case study for lactoferricin derived peptides. BMC Microbiol 8:196.
24. Guzman L M, Belin D, Carson M J, Beckwith J. 1995. Tight regulation, modulation, and high-level expression by vectors containing the arabinose P_{BA}D promoter. J Bacteriol 177:4121-4130.
25. Lood R, Raz A, Molina H, Euler C W, Fischetti V A. 2014. A highly active and negatively charged Streptococcus pyogenes lysin with a rare D-alanyl-L-alanine endopeptidase activity protects mice against streptococcal bacteremia. Antimicrob Agents Chemother 58:3073-3084.
26. CLSI. 2015. Methods for Dilution Antimicrobial Susceptibility Tests for Bacteria That Grow Aerobically; Approved Standard-10th Edition. Clinical and Laboratory Standards Institute, Wayne, Pa.
27. Ceri H, Olson M E, Stremick C, Read R R, Morck D, Buret A. 1999. The Calgary Biofilm Device: new technology for rapid determination of antibiotic susceptibilities of bacterial biofilms. J Clin Microbiol 37:1771-1776.
28. Schuch R, Khan B K, Raz A, Rotolo J A, Wittekind M. 2017. Bacteriophage Lysin CF-301, a Potent Antistaphylococcal Biofilm Agent. Antimicrob Agents Chemother 61.
29. Moody J. 2010. Synergy testing: broth microdilution checkerboard and broth macrodilution methods, p 5.12.11-15.12.23. In Garcia L S (ed), Clinical Microbiology Procedures Handbook, vol 2. ASM Press, Washington, D.C.
30. Lv Y, Wang J, Gao H, Wang Z, Dong N, Ma Q, Shan A. 2014. Antimicrobial properties and membrane-active mechanism of a potential alpha-helical antimicrobial derived from cathelicidin PMAP-36. PLoS One 9:e86364.
31. Scanlon T C, Teneback C C, Gill A, Bement J L, Weiner J A, Lamppa J W, Leclair L W, Griswold K E. 2010. Enhanced antimicrobial activity of engineered human lysozyme. ACS Chem Biol 5:809-818.
32. Teneback C C, Scanlon T C, Wargo M J, Bement J L, Griswold K E, Leclair L W. 2013. Bioengineered lysozyme reduces bacterial burden and inflammation in a murine model of mucoid Pseudomonas aeruginosa lung infection. Antimicrob Agents Chemother 57:5559-5564.
33. Griswold K E, Bement J L, Teneback C C, Scanlon T C, Wargo M J, Leclair L W. 2014. Bioengineered lysozyme in combination therapies for Pseudomonas aeruginosa lung infections. Bioengineered 5:143-147.
34. Daniels D S, Schepartz A. 2007. Intrinsically cell-permeable miniature proteins based on a minimal cationic PPII motif. J Am Chem Soc 129:14578-14579.
35. Vaara M, Porro M. 1996. Group of peptides that act synergistically with hydrophobic antibiotics against gram-negative enteric bacteria. Antimicrob Agents Chemother 40:1801-1805.
36. Briers Y, Walmagh M, Van Puyenbroeck V, Cornelissen A, Cenens W, Aertsen A, Oliveira H, Azeredo J, Verween G, Pirnay J P, Miller S, Volckaert G, Lavigne R. 2014. Engineered endolysin-based “Artilysins” to combat multidrug-resistant gram-negative pathogens. MBio 5:e01379-01314.
37. Briers Y, Walmagh M, Grymonprez B, Biebl M, Pirnay J P, Defraine V, Michiels J, Cenens W, Aertsen A, Miller S, Lavigne R. 2014. Art-175 is a highly efficient antibacterial against multidrug-resistant strains and persisters of Pseudomonas aeruginosa. Antimicrob Agents Chemother 58:3774-3784.

1.-10. (canceled)

11. A method of resensitizing an antibiotic-resistant Gram-negative bacteria to an antibiotic, the method comprising contacting the antibiotic-resistant Gram-negative bacteria with (i) an antibiotic and (ii) a pharmaceutical composition containing (a) a modified Gram-negative lysin polypeptide wherein the charged amino acid residues of native lysin proteins are mutagenized randomly by noncharged amino acid residues; (b) a native Gram-negative lysin polypeptide, wherein said modified or native Gram-negative lysin polypeptide is optionally fused to an antimicrobial peptide (AMP) sequence with or without a linker; or (c) fragments thereof having lytic activity or variants thereof having lytic activity and having at least 80% sequence identity with said modified or native Gram-negative lysin polypeptide, in an amount effective to resensitize the antibiotic-resistant Gram-negative bacteria to the antibiotic.

12. A method of treating a bacterial infection caused by an antibiotic-resistant Gram-negative bacteria, comprising administering to a subject diagnosed with, at risk for, or exhibiting symptoms of a bacterial infection, (ii) an antibiotic and (ii) a composition containing (a) a modified Gram-negative lysin polypeptide wherein the charged amino acid residues of native lysin proteins are mutagenized randomly by noncharged amino acid residues; (b) a native Gram-negative lysin polypeptide, wherein said modified or native Gram-negative lysin polypeptide is optionally fused to an antimicrobial peptide (AMP) sequence with or without a linker; or (c) fragments thereof having lytic activity, or variants thereof having lytic activity and having at least 80% sequence identity with said lysin polypeptide, in an amount effective to resensitize the antibiotic-resistant Gram-negative bacteria to the antibiotic.

13. The method of claim 12, wherein the antibiotic-resistant Gram-negative bacteria is selected from the group consisting of Pseudomonas aeruginosa, Klebsiella spp., Enterobacter spp., Escherichia coli, Citrobacter freundii, Salmonella typhimurium, Yersinia pestis, and Franciscella tulerensis.

14. The method of claim 12, wherein the antibiotic-resistant Gram-negative bacteria is Pseudomonas aeruginosa.

15. The method of claim 12, wherein the bacterial infection is a topical or systemic pathogenic bacterial infection.

16. (canceled)

17. The method of claim 12, wherein the antibiotic is selected from one or more of ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B, and colistin.

18.-22. (canceled)

23. A method according to claim 11, wherein said antibiotic-resistant Gram-negative bacteria are in a biofilm, the method effecting disruption of the biofilm.

24. (canceled)

25. (canceled)

26. The method of claim 12, wherein the antibiotic is a carbapenem.

27. The method of claim 12, wherein the antibiotic-resistant Gram-negative bacteria are in a biofilm, the method effecting disruption of the biofilm.

28. The method of claim 11, wherein the antibiotic-resistant Gram-negative bacteria is selected from the group consisting of Pseudomonas aeruginosa, Klebsiella spp., Enterobacter spp., Escherichia coli, Citrobacter freundii, Salmonella typhimurium, Yersinia pestis, and Franciscella tularensis.

29. The method of claim 11, wherein the antibiotic-resistant Gram-negative bacteria is Pseudomonas aeruginosa.

30. The method of claim 11, wherein the antibiotic is selected from one or more of ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B, and colistin.

31. The method of claim 11, wherein the antibiotic is a carbapenem.

32. The method of claim 11, wherein the lysin polypeptide is GN121 or GN123.

33. The method of claim 12, wherein the lysin polypeptide is GN121 or GN123.

34. The method of claim 11, wherein the modified or native Gram-native lysin polypeptide is selected from GN121, GN123, GN202, GN147, GN146, GN156, GN92, GN54, GN201, GN94, GN200, GN204, GN205, GN3, GN13, GN17, GN9, GN10, GN105, GN108, GN150, GN203, GN4, GN37, or a fragment thereof having lytic activity, and variants thereof having lytic activity and having at least 80% sequence identity with said lysin polypeptide.

35. The method of claim 12, wherein the modified or native Gram-native lysin polypeptide is selected from GN121, GN123, GN202, GN147, GN146, GN156, GN92, GN54, GN201, GN94, GN200, GN204, GN205, GN3, GN13, GN17, GN9, GN10, GN105, GN108, GN150, GN203, GN4, GN37, or a fragment thereof having lytic activity, and variants thereof having lytic activity and having at least 80% sequence identity with said lysin polypeptide.
